SEQUENCE LISTING (21 sequences)

SEQ ID NO: 1 - DNA, 30 bp, Homo sapiens
ccgacctgttctggaggacggtaacctcag

SEQ ID NO: 2 - DNA, 25 bp, Homo sapiens
accaggggctgcagccagccctatc

SEQ ID NO: 3 - DNA, 26 bp, Artificial Sequence
  A synthetic primer with homology to the pBKS II cloning vector.
ggaaacagctatgaccatgattacgc

SEQ ID NO: 4 - DNA, 30 bp, Homo sapiens
gaccggagccgagctcggggttgctcgagg

SEQ ID NO: 5 - DNA, 30 bp, Homo sapiens
atctctggacctgcctcttggtcattacgg

SEQ ID NO: 6 - DNA, 30 bp, Mus musculus
gggtcctctggagctggagttacagactac

SEQ ID NO: 7 - DNA, 29 bp, Mus musculus
gcttggcttaagatacagcagctatcctg

SEQ ID NO: 8 - DNA, 30 bp, Homo sapiens
cacgcacccaaattagaagctgctgggtcg

SEQ ID NO: 9 - DNA, 30 bp, Homo sapiens; identical to SEQ ID NO: 1

SEQ ID NO: 10 - DNA, 30 bp, Homo sapiens
cacacgggaagctggactctggccatctcg

SEQ ID NO: 11 - DNA, 30 bp, Homo sapiens
aaaccctggccagacctggagttcaggagg

SEQ ID NO: 12 - DNA, 30 bp, Homo sapiens
aacctccccctcccaaaggctatggagagc

SEQ ID NO: 13 - DNA, 30 bp, Homo sapiens
tgcgcgtgtggcctggcatgaggtatgtcg

SEQ ID NO: 14 - DNA, 30 bp, Homo sapiens
gtgctgggccctgacccctccacgtctgtc

SEQ ID NO: 15 - DNA, 10 bp, Artificial Sequence
  transgene/genome junction
tcctgctgcc

SEQ ID NO: 16 - DNA, 35 bp, Mus musculus
catctagcttatattcccagtatctttgaagtttt

SEQ ID NO: 17 - DNA, 35 bp, Unknown
  5′ transgene junction
catctagcttatattcccagctgaggtgcttgctg

SEQ ID NO: 18 - DNA, 29 bp, Unknown
  Injected DNA
taaaaaactacaaccagctgaggtgcttg

SEQ ID NO: 19 - DNA, 35 bp, Mus musculus
caaactcagaaatcctcctgcctctgcctcccgag

SEQ ID NO: 20 - DNA, 35 bp, Unknown
  3′ transgene junction
cacaagagggactcctgctgcctctgcctcccgag

SEQ ID NO: 21 - DNA, 29 bp, Unknown
  Injected DNA
aagagggactcctgctgcccaccaggcat

(19) United States
(12) Patent Application Publication
(10) Pub. No.: US 2008/0289059 A1
(43) Pub. Date: Nov. 20, 2008

(54) Methods for developing animal models

(76) Inventor: Tatsuji Nomura, Tokyo (JP)

Correspondence Address: Goodwin Procter LLP;Attn: Patent Administrator, 135 Commonwealth Drive, Menlo Park, CA, 94025-1105, US

(21) Appl. No.: 12/172,090

(22) Filed: Jul. 11, 2008

Related U.S. Application Data
(63) Continuation of application No. 10/179,639, filed on Jun. 24, 2002.

(51) Int. Cl. A01K 67/027

(52) U.S. Cl. 800/18

(57) ABSTRACT

The invention concerns methods for the development of mutant animals, including genetically engineered animals and those carrying spontaneous mutations, as human disease models. In particular, the invention provides an integrated technology, including rigorous specifications and quality control, for the development of animal models that can serve as a living assay system, useful in biomedical research and in the development of human therapeutics.

### CROSS REFERENCE TO RELATED APPLICATIONS

This application is a continuation of U.S. patent application Ser. No. 10/179,639 filed on Jun. 24, 2002, which application is fully incorporated herein by reference.

### BACKGROUND OF THE INVENTION

1. Field of the Invention

The invention concerns methods for the development of mutant animals, including genetically engineered animals and those carrying spontaneous mutations, as human disease models. In particular, the invention provides an integrated technology, including rigorous specifications and quality control, for the development of animal models that can serve as a living assay system, useful in biomedical research and in the development of human therapeutics.

2. Description of the Related Art

Mutant animals, including genetically engineered animals, such as transgenic mice, and animals with spontaneous mutations, initially served as animal models in the field of molecular biology. In recent years, the use of such animals has been extended to many other branches of life sciences, including the identification and study of disease related genes, and drug development targeting such genes.

Although more than 10,000 kinds of gene manipulated animals, such as transgenic mice, knock-out mice and knock-in mice have been created and widely used by basic researchers in bioscience in the past two decades, an overwhelming majority of genetically engineered animals have serious deficiencies as research tools and tools of drug development. In most cases, the producers of genetically engineered animals fail to subject the animals to a thorough, rigorous and reliable validation process, and, as a result, cannot ensure that the animals are identical both in genetic and in microbiological aspects. This is a serious problem since the genetic background of transgenic animals, along with differences in their exposure to environmental factors, has a large effect on their behavior in vivo. Every single genetic or environmental difference results in dramatic differences in the overall characteristics of the genetically engineered animals. Furthermore, because the selection and determination of genetic and microbiological control based on expert knowledge are typically not performed by experts who are knowledgeable about the subject human disease, the usefulness of genetically engineered animals as reliable disease models is limited.

### SUMMARY OF THE INVENTION

In one aspect, the invention concerns a method of establishing a mutant animal line, comprising the steps of:

(a) inducing superovulation in a sexually immature mutant founder animal (GO);

(b) fertilizing the superovulating sexually immature mutant founder animal;

(c) delivering a first generation mutant animal (F1) upon completion of the gestation period;

(d) confirming stability of the mutation, genotype, and identity of genetic background in the first generation mutant animal; and, if desired,

(e) repeating steps (a)-(d) with one or more further generations of mutant animals, wherein in each step the genetic and environmental factors are monitored and kept strictly identical for all animals.

In one embodiment, fertilization is performed by natural mating.

In another embodiment, fertilization is performed by
(b. 1) subjecting an oocyte obtained from the superovulating premature mutant founder animal to in vitro fertilization;
(b. 2) culturing the fertilized oocyte in vitro to an early embryonic stage; and
(b. 3) introducing the embryo into a recipient animal.

In a preferred embodiment, in step (b. 2) above, the fertilized oocyte is cultured to a two-cell embryonic stage. The early embryo may be stored in an embryo bank prior to introduction into a recipient animal, typically at liquid nitrogen temperature.

The invention is not limited to any particular mutant animal, and specifically includes, without limitation, all non-human mutant mammals, including mice, rats, rabbits, cats, dogs, guinea pigs, and other animals typically used in laboratory experiments. The preferred mutant animal is a mutant mouse, including transgenic, knock-in, knock-out and spontaneous mutant mice. In a preferred embodiment, the mutant animal is a transgenic mouse.

In a typical, but not limiting, protocol, the founder animal is three to four weeks old at the time of achieving superovulation. Superovulation can be induced by any conventional method, including, for example, the use of pregnant mere serum gonadotrophin (PMSG) and human chorionic onadotropin (hCG).

In a preferred embodiment, in step (d) of the foregoing method, genotype is determined by

(d1) performing a PCR reaction on genomic DNA isolated from transgenic and corresponding non-transgenic animals, using the following PCR primers: (i) a chromosome specific primer and a transgene specific primer binding, in opposite directions, to the chromosome and the transgene near the 5′transgene/genome junction, for verification of the 5′transgene/genome junction; and (ii) two transgene specific primers binding, in opposite direction, to a segment of the transgene near the 5′end for verification of transgene/transgene junctions,

(d2) separating of the amplified PCR products by size, and

(d3) determining genotype based on the size pattern of the amplified PCR products indicating the copy number of the integrated transgene.

The method can further comprise the use, in step (d1), of a transgene specific primer and a chromosome specific primer binding, in opposite directions, to the transgene and the genome near the 3′transgene/genome junction, for verification of the 3′transgene/genome junction.

In another embodiment, the method can further comprising the use, in step (d1) of two chromosome specific primers binding, in opposite directions, to the chromosome near to a chomosome/transgene junction, for verification of the pre-integration site.

In a typical protocol, each generation of the mutant, e.g. transgenic, animals is subjected to scheduled genetic monitoring and spot checks. Preferably, genetic monitoring includes monitoring of one or more genes in the genetic background.

Preferably, each generation of the mutant animals is subjected to scheduled monitoring and spot checks of environmental factors, where the environmental factors include factors of the developmental and proximate environment. Most preferably, only animals having the same genotype, phenotype and dramatype as the founder animal are included in the production of further generations of mutant, e.g. transgenic, animals.

In another aspect, the invention concerns mutant animals produced by the foregoing method. The mutant animal can, for example, be a transgenic mouse, such as a Tg-rasH2 mouse, carrying the human c-Ha-ras transgenic, or a TgPVR21 mouse, carrying the human poliovirus receptor (PVR) gene.

### BRIEF DESCRIPTION OF THE DRAWINGS

FIG. 1 is a graphic illustration of the main factors which influence the results of animal experiments.

FIG. 2 illustrates the control of genetic and environmental factors in accordance with the invention.

FIG. 3 shows the factors controlled as part of the quality assurance test in developing the animal experimentation system of the invention.

FIG. 4 is a graphical illustration of the “super speed” congenic method of the invention.

FIG. 5 illustrates two types of genetic quality testing, depending on the aim.

FIG. 6 illustrates the genotyping of a transgenic animal by duplex PCR.

FIG. 7 shows the chromosomal localization of the integrated transgene in Tg-rasH2 mice at N15 and N20 as determined by the FISH method. A paired fluorescent signal representing the transgene location was observed on the chromosome 15E3 region in both eases. The Tg-rasH2 mouse is a hemizygote, so the hybridization signal was only detected in one pair of sister chromatids.

FIG. 8 shows the results of Southern blot analysis of transgene integration in Tg-rasH2 mice. (A) Restriction map and structure of the transgene in Tg-rasH2 mice (7.0-kb BamHI fragment of human c-Ha-ras gene). The open boxes represent four exons (Ex1 to Ex4) encoding a human c-Ha-ras protein. The DIG-labeled 5′-probe recognizing the upstream region from XbaI was indicated as an open circle and bar. (B) Genomic DNA from non-transgenic and Tg-rasH2 mice at N15 and N20 was BamHI digested, electrophoresed on 0.6% agarose gel, and transferred to nylon membranes. The membrane was hybridized with DIG-labeled random primed probe. DNA samples were obtained from a non-transgenic mouse (lane 1), Tg-rasH2 mice at N15 (lanes 2 and 3) and Tg-rasH2 mice at N20 (lanes 4 and 5). (C) Genomic DNA from a Tg-rasH2 mouse at N20 was restriction endonuclease digested (lane 1; BamHI, 2; HpaI, 3; XhoI, 4; XbaI, 5; NcoI, 6; BglII, 7; Sad, 8; HindIII), electrophoresed on 0.6% agarose gel, and transferred to nylon membranes. The membrane was hybridized with a DIG-labeled 5′-probe. The signal was detected with chemiluminescent alkaline phosphatase substrates and on an X-ray film.

FIG. 9 shows the results of Northern blot analysis integration in Tg-rasH2 mice. Expression of human c-Ha-ras mRNA in a Tg-rasH2 mouse at N15 and N20. Ten-microgram samples of total RNA (B, L and F indicate brain, lung and forestomach, respectively) were fractionated on formalin-agarose gel and transferred to a nylon membrane. The membrane was hybridized with [a-32P]-dCTP labeled human c-Ha-ras gene (c-Ha-ras) probe, and then rehybridized with [a-32p]-dCTP labeled human Glyceraldehyde-3-phosphate dehydrogenase cDNA (GAPDH) probe. nTg and Tg indicate the samples obtained from non-transgenic and Tg-rasH2 mice, respectively. The signal was detected on X-ray film.

FIG. 10 shows the results of Southern blot hybridization for determination of the exact copy number of the integrated human c-Ha-ras gene in the Tg-rasH2 mouse. Genomic DNA from a Tg-rasH2 mouse at N20 was completely digested with BamHI (lane 1) and Hindi (lane 2). Hindi digested genomic DNA was further digested with various concentrations of BamHI (lane 3-5). The digested DNA was then electrophoresed on 0.4% agarose gel and transferred to a nylon membrane. The membrane was hybridized with a DIG-labeled random primed probe. The signal was detected with chemiluminescent alkaline phosphatase substrates and on an X-ray film.

Lane marked M, Expand TM DNA molecular weight marker (Roche Diagnostics GmbH).

FIG. 11 illustrates the results of PCR verification of genome/transgene junctions. (A) PCR was performed on genomic DNA from Tg-rasH2 (T) and non-transgenic (N) mice with the following primer sets: for verification of the 5′ genome/transgene junction; chromosome specific primer A and transgene specific primer C, for verification of the 3′transgene/genome junction; transgene specific primer D and chromosome specific primer B, for identification of pre-integration site; chromosome specific primer A and B, and for verification of transgene/transgene junctions; transgene specific primer D and C. (B) The PCR product created using D and C primers was digested with restriction endonuclease BamHI to confirm the integrity of transgene/transgene junctions. A 100-bp DNA ladder was used as a DNA size marker, (C) Three transgenes at the interrupted locus in the mouse genome. The human c-Ha-ras transgene is present in a head-to-tail tandem array (solid boxes indicate exons). Arrowheads depict both the position and direction of the oligonucleotides used, with the tip of the arrow representing the 3′ end of the oligonucleotide.

FIG. 12 shows the result of sequence analysis of the genome/transgene junctions in a Tg-rasH2 mouse. The corresponding regions in non-transgenic mouse DNA and injected DNA are also shown for comparison. Asterisks indicate identical nucleotide, and boxed areas denote identity with the nucleotide at the site of recombination. Horizontal arrows represent the Topoisomerase I consensus sequence (5′-A/T-G/C-T/A-T-3′).

FIG. 13 illustrates the embryo banking facility with respect to microbiological control and planned production.

FIG. 14 is an illustrative explanation of the Alternative Microbiological Control Method.

FIG. 15 is a schematic illustration of the Planned Production and Supply System of the invention.

FIG. 16 shows the results of FISH analysis and chromosomal localization of integrated PVR transgene in TgPVR21 mice.

FIG. 17 shows the results of Southern blot analysis of the PVR transgene in TgPVR21 mice.

FIG. 18 shows the results of Northern blot, RT-PCR and direct sequencing analysis in order to determine the gene expression profile of PVR mRNA in TgPVR21 mice.

FIG. 19 shows the structure of PVR-a,- (3, and -y mRNA, and the sites of probe, primers and sequencing.

FIG. 20 shows the structure of 5′genome/transgene junction in a TgPVR21 transgenic mouse.

FIG. 21 shows the restriction map of the 5′genome/transgene junction in a TgPVR21 transgenic mouse.

FIG. 22 shows the results of sequencing the 5′genome/transgene junction in a TgPVR21 transgenic mouse.

FIG. 23 illustrates the determination of the structure of upstream site of the transgene/mouse genome junction region in TgPVR21 mouse relative to Clone No. 2833685.

FIG. 24 is a graphical illustration of the production and validation system of the invention.

FIG. 25 shows tumor incidence for N-Methyl-N-nikosourea (U) positive controls; forestomach papilloma (single i. p./75 mg/kg).

FIG. 26 shows tumor incidence for MNU prosive controls; malignan lymphoma (single i. p./75 mg/kg).

Table A is chart showing materials and methods used in experiments for the present application.

### DETAILED DESCRIPTION OF THE PREFERRED EMBODIMENT

### A. Definitions

The term “mutant” animal is used in the broadest sense, and specifically includes genetically engineered (gene manipulated) animals, such as transgenic, knock-out and knock-in animals, and animals carrying spontaneous mutations and mutations generated by artificial mutagenesis in one or more genes.

The terms “genetically engineered” and “gene manipulated” are used interchangeably, and refer to transgenic, knock-in and knock-out animals.

As used herein, the term “egg,” when used in reference to a mammalian egg, means an oocyte surrounded by a zona pellucida and a mass of cumulus cells (follicle cells) with their associated proteoglycan. The term “egg” is used in reference to eggs recovered from antral follicles in an ovary (these eggs comprise pre-maturation oocytes) as well as to eggs which have been released from an antral follicle (a ruptured follicle).

The term “oocyte.” as used herein, refers to a female gamete cell and includes primary oocytes, secondary oocytes and mature, unfertilized ovum. An oocyte is a large cell having a large nucleus (i.e., the germinal vesicle) surrounded by ooplasm. The ooplasm contains non-nuclear cytoplasmic contents including mRNA, ribosomes, mitochondria, yolk proteins, etc. The membrane of the oocyte is referred to herein as the “plasma membrane.”

The term “hemizygote” with reference to a transgenic animal means that the transgenic animal carries haploid of the wild-type gene and haploid of the transgene (or haploid of the set of transgenes when more than one copy of the transgene is integrated).

The term “gonosome” is used to refer to a sex chromosome. In mammals, the X and Y chromosomes determine the sex of an individual. Females have two X chromosomes, while males have one X and one Y chromosomes The term “hemizygous” as used with reference to a genetically modified, such as transgenic, animal herein applies to being an hemizygote for the gene referred to, such as transgene.

The term “homozygote” refers to a diploid genotype in which the two alleles for a given genes are identical. With reference to a transgenic animal, the term means that the animal carries diploid of the transgene (or diploid of the set of transgenes when more than one copy of the transgene is integrated).

The term “heterozygote” refers to a diploid genotype in which the two alleles for a given gene are different.

The term “transgenic animal” is used to refer to an animal which is altered by the introduction of recombinant DNA through human intervention. This includes animals with heritable germline DNA alterations, and animals with somatic non-heritable alterations. The term “transgene” refers to a nucleic acid (DNA) which is either (1) introduced into somatic cells or (2) integrated stably into the germ line of its animal host strain, and is transmissible to subsequent generations.

The term “genotype” refers to the “internally coded, inheritable information” carried by all living organisms. This stored information is used as a “blueprint” or set of instructions for building and maintaining a living creature. These instructions are found within almost all cells (the “internal” part), they are written in a coded language (the genetic code), they are copied at the time of cell division or reproduction and are passed from one generation to the next (“inheritable”). These instructions are intimately involved with all aspects of the life of a cell or an organism.

The “genotype” controls everything from the formation of protein macromolecules, to the regulation of metabolism and synthesis.

The term “phenotype” refers to the “outward, physical manifestation” of the organism. These are the physical parts, the sum of the atoms, molecules, macromolecules, cells, structures, metabolism, energy utilization, tissues, organs, reflexes and behaviors; anything that is part of the observable structure, function or behavior of a living organism.

The term “dramatype” refers to the pattern of performance in a single physiological response of an experimental animal. Variation in such responses is the joint product of two factors: the phenotype itself, and the proximate environmental conditions in which the animals are tested, such as, temperature, humidity, diet, investigators and animal care personnel, etc. For uniform dramatype, the environmental conditions in which the animals are tested must be strictly controlled.

The term “congenic animal” refers to animal strains that are produced by repeated back-crosses to an inbred (background) strain, with selection for a particular marker from the donor strain.

The term “hybrid animal” refers to animals, e.g. mice or rats that are the progeny of two inbred strains, crossed in the same direction, are genetically identical, and can be designated using upper case abbreviations of the two parents (maternal strain listed first), followed by F1.

The term “scheduled monitoring” refers to examination that is performed regularly and by a standard method in order to assure that the genetic and microbiological quality of the already defined animal is stable through time. This is done by comparing the genetic and microbiological profiles of the defined mice and the corresponding inbred strains. This scheduled monitoring gives information not only of the maintenance of animal health but also about maintenance of the specified quality.

Conceptually, experimental animals can be viewed as “living measurement tools.” They are unique in that, contrary to tools used in physicochemical measurements, they are changing day by day. Therefore, monitoring the quality of experimental animals is essential for their intended use. This need does not exist in connection with pets or farm animals.

The term “spot checking” refers to the unscheduled examination of animals that is performed at irregular intervals to determine whether the animals have been subject to any infection or genetic contamination.

The term “two layer monitoring” refers to a monitoring system combining scheduled monitoring and spot checking.

The term “gnotobiote” is used to refer to animal strains derived by aseptic surgical procedures or from sterile hatching of eggs, which are reared and maintained with germfree techniques under isolator conditions and in which the composition of the associated fauna and flora, if present, is fully defined by accepted current methodology.

### B. Detailed Description

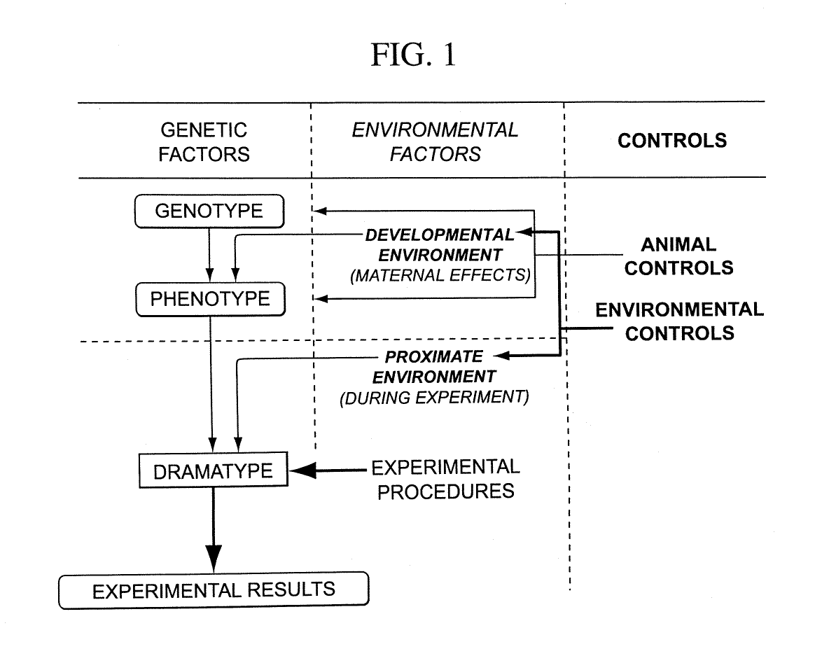

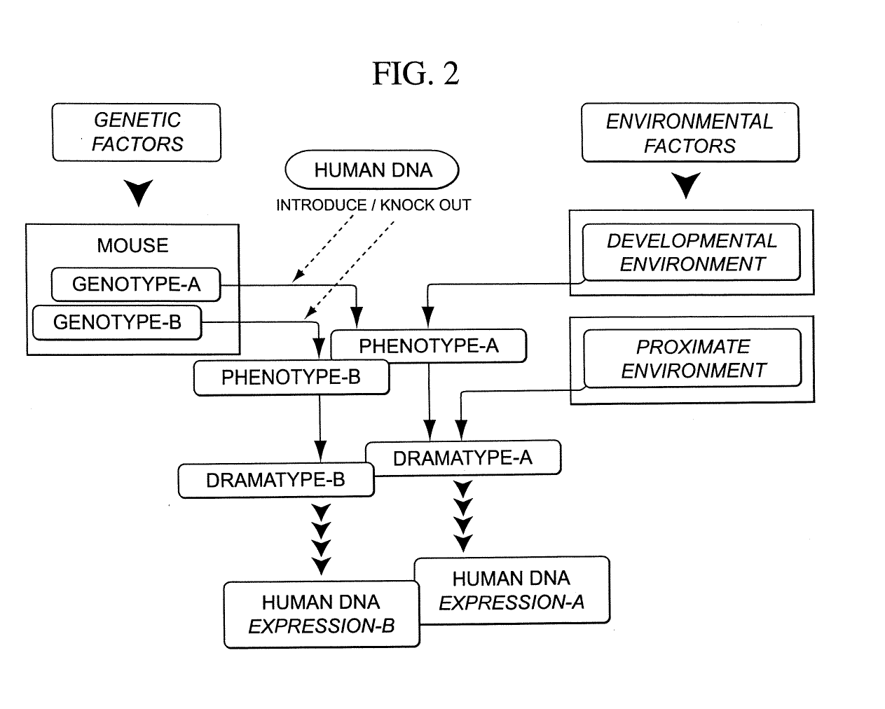

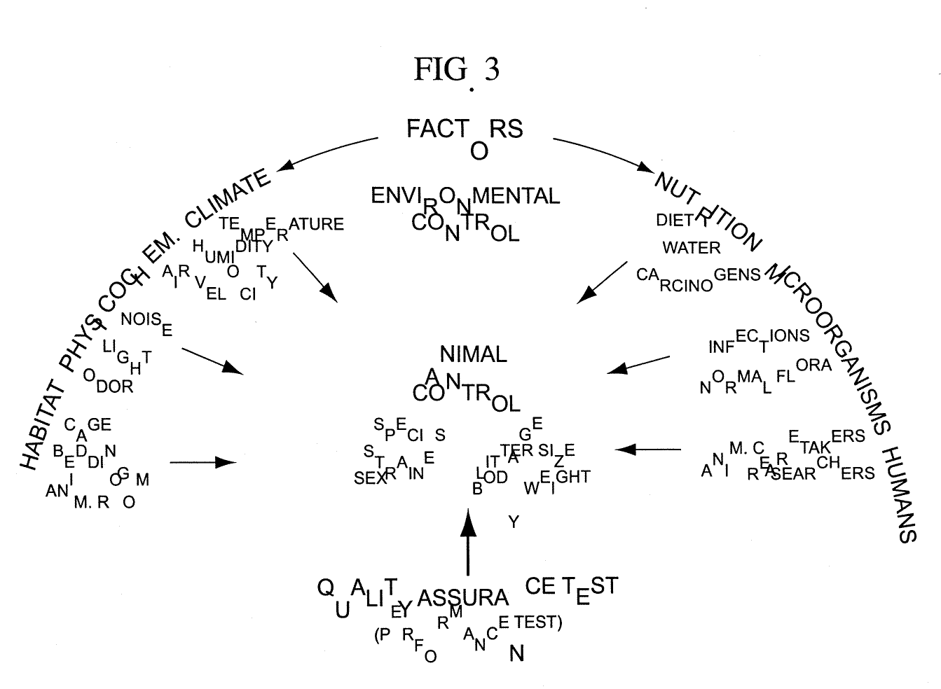

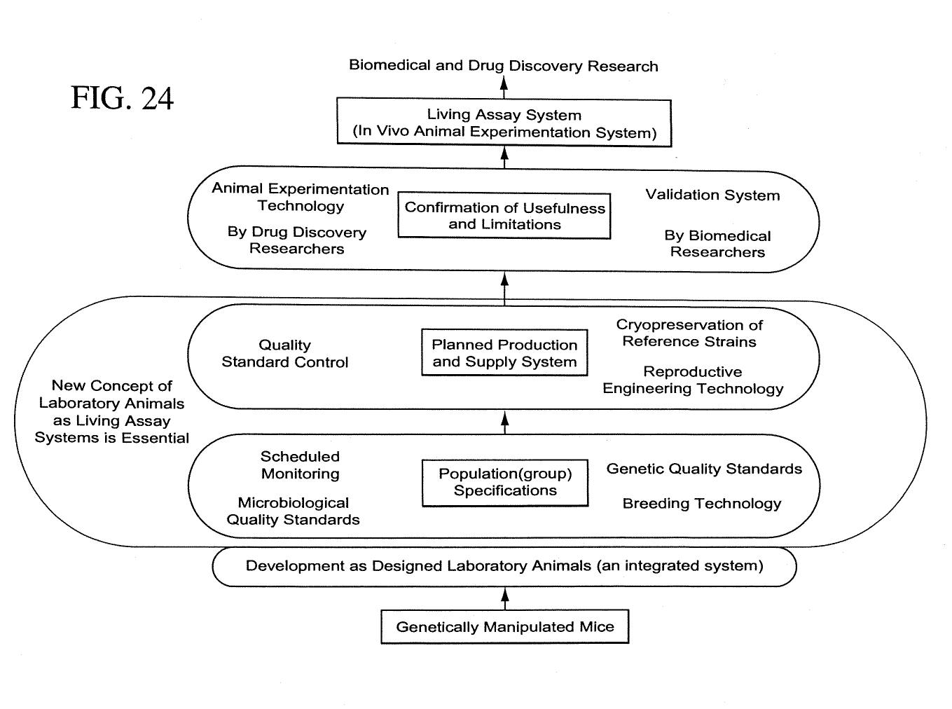

The present invention concerns an integrated production and supply system for the design and development of mutant, such as genetically engineered animals or animals with spontaneous mutations, that can be used as reliable tools in biomedical research and drug development. As noted before, it is imperative that such mutant experimental animals be completely identical in all of their properties, since even the slightest differences in various genetic and/or environmental factors dramatically influence the outcome of animal experiments. Specifically, all animals must have the same genotype, phenotype, and dramatype, and must be subject to the same developmental environment (maternal effects), and proximate environment. These factors, which significantly influence the results of animal experiments are illustrated in FIG. 1. In order to achieve this, a new production and validation system has been developed for the development of mutant, e.g. genetically engineered, animal models that can be viewed as a living assay system (in vivo experimentation system), just as reproducible as well-established physicochemical assay systems. The new production and validation system is graphically illustrated in FIG. 24. In this system, all factors affecting the properties of the animals are tightly controlled. This includes tight control of the animals themselves (e.g., species, strain, and their combination in breeding to create hybrid animals, sex, age, litter size, and bodyweight), their habitat (e.g., bedding, animal room, etc.), physicochemical factors (e.g. odor, light, noise), climate (e.g., air velocity, humidity, temperature), nutrition (e.g., diet, water, exposure to carcinogens), microorganisms (e.g., infections, quality of normal flora), and human factors (e.g., animal caretakers, researchers, etc.). In addition, the balance between the genetically controlled background strain and the genetic diversity has been designed, selected, and determined by experts in experimental animal science and an expert for the subject animal disease. These factors are graphically illustrated in FIG. 2.

The development of standardized laboratory animals that can be used as “living test instruments,” begins, as a first step, with the preparation of reference animals complying with uniform genetic and microbiological quality specifications, followed, as a second step, by the establishment of a planned production system so that sufficient numbers of identical animals can be obtained to evaluate their usefulness and limitations in any particular animal experiment. Hundreds or even thousands of standardized animals can be developed following this approach, and subjected to thorough evaluation, to determine their usefulness and limitations as a model system before a genetically engineered animal model is established. An important third step in die practical development of standardized genetically engineered animal models involves the establishment of an integrated “in vivo experimentation system,” using well-characterized, reliable animal models so that the genetically engineered animals, such as transgenic mice, can be reliably used as human disease models or in other fields of biomedical and pharmaceutical research. Typically, laboratory animal scientist assume responsibility for the first step, and medical or pharmaceutical researchers using the animal models accomplish the third step, while both groups of researchers participate in the second step. While this system will be illustrated with reference to transgenic animals, the invention is not so limited. The approach of the present invention is equally applicable to other mutant animals, including all types of genetically engineered (gene manipulated) animals and animals carrying one or more spontaneous mutations.

Step 1: Preparation of Reference Animals with Uniform Genetic and Microbiological Quality Standards

Traditionally, the development of transgenic animals includes the following steps: (1) introduction of DNA into mouse eggs by microinjection, or into embryonic stem cells (ES cells) by retroviral vectors or by other methods; (2) testing by reliable genotyping assays to confirm that the transgene has been integrated and transmitted, e.g., by PCR or Southern blot (founder animals); (3) reproduction by cloning or by sexual reproduction; and (4) quality control, including genetic quality control (genetic profile monitoring) by using biochemical markers and microbiological quality control, followed by microbiological monitoring system. Since even small changes can yield critical differences in how the animals behave in the laboratory experiments, monitoring and quality assurance of each step, as well as excellence in maintaining a breeding colony, are essential for the reliability of mutant, e.g. genetically engineered, animal models. The present invention provides significant improvements in each step of this overall process.

In particular, the present invention provides for population specification, including (1) genetic quality assurance (super speed congenic method) and (2) microbiological assurance (two-layer monitoring) in the process of developing laboratory animals for in vivo experiments. This part of the breeding process will be referred to as the “population stage.” In addition, the invention provides for a “planned production and supply system”, which includes (1) ongoing monitoring of mutant, e.g. transgene, stability and function, (2) a risk management system (bulk preservation), (3) reproductive engineering technology, and (4) selection of background strain for specific aims of the model and, if necessary, widening the genetic background in order to achieve widened, but repeatable and reproducible genetic diversity.

Genetic Quality Assurance (SuPer sPeed Method to Develop Convenic Animals)

The genetic quality assurance step of the present invention includes the preparation and validation of reference mutant, such as, transgenic animals, e.g. mice, with uniform genetic and microbiological quality standards, and a speed congenic process and techniques, which are followed by genetic monitoring on a scheduled basis, to ensure that the required qualities are being maintained. In this step, the present invention assures not only proper insertion of the transgene, in the case of transgenic animals, but also the identity of background genes of the mouse or other mutant animal. The importance of assuring that the genetic background is also identical in the mutant animal is that in the absence of a 100% identity in the genetic background, the mutant animal might loose the phenotype of the parental animal. For example, p53+/−mice have shown complicated phenotype due to this reason. The major improvements provided by the this step of the invention are speeding up the process, i.e. shortening the time necessary to establish congenic animals, and the application of a two layer genetic monitoring system.

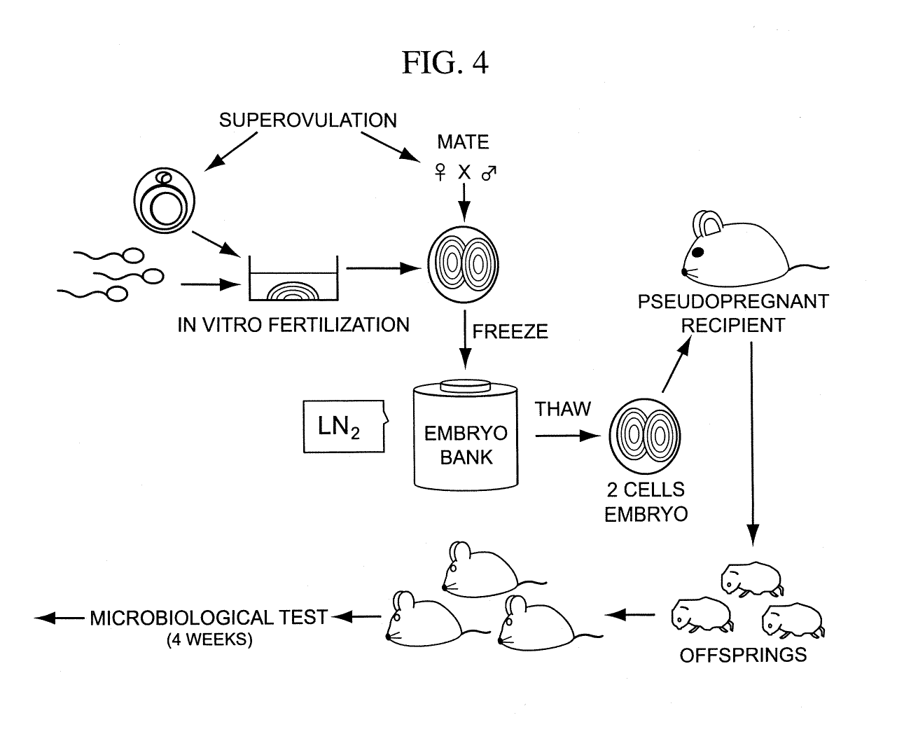

The development of new mutant, such as, transgenic, knock-out or knock-in, animal lines typically requires careful back-crossing for at least 5 generations, more frequently at least 9 generations, often for up to 12 generations to establish the genetic manipulation or spontaneous mutation, on a particular in-bred animal, such as mouse strain. The result of this process is the establishment, after several generations, of a mutant, e.g. transgenic, knock-out or knock-in animal model, on a fixed genetic background, referred to as “congenic.” The animals subjected to in vitro fertilization are typically at least about two months old, and the pregnancy period is 19 days, which means that the production of each generation takes almost three months. As a result, the establishment of a congenic animal strain is a very long process, which typically takes years. The present invention provides a high-speed method for establishing a congenic animal strain. According to the invention, female animals, e.g. mice, of each generation are treated to overovulate, and subjected to in vitro fertilization at the young age of approximately four weeks. Usually 16-day old female mice are injected by PMSG followed by hCG injection at day 28 to achieve superovulation. On day 20, the mice are mated naturally or subjected to in vitro fertilization. On day 30, the two cells embryos are collected and transplanted into pseudopregnant recipients. Since the pregnancy period is 19 days, delivery takes place on day 49. It is easy to see that the use of atypical young (about four weeks) animals in each generation significantly reduces the time required for the establishment of a congenic strain. This process is graphically illustrated in FIG. 4, and described in greater detail in Example 1. A similar process can be used to produce congenic animals from strains showing low potency of ovulation.

Microbiolovical Quality Assurance-Alternative Microbiolovical Quality Control Method

The microbiological environment is one of the main factors that influence the dramatype of laboratory animals. It is well known fact that outbreaks of microbial infections alter the health of laboratory animals and, as a result, the experimental results such as performance of reproduction, and blood chemistry (Nomura, T. Genetic and microbiological control. In; Immune-Deficient Animals (Sordat, B. et al eds.), S. Karger A G. Basel 1984; Itoh, T. et al. Expr. Anim., 30: 491-495, 1981; Itoh. T. et al. Jpn. J. Vet. Sci. 40: 615-618.1978; Iwai. H. et al., Expr. Anim. 26: 205-212, 1977). Furthermore, Narushima et al. (Narushima et al., Exp Anim 47 (2): 111-7 (1998)) demonstrated that intestinal bacteria modified response to carcinogens in the transgenic rasH2 mice. These findings strongly indicate that strict control of microbiological environment is indispensable for the assurance of dramatypical quality of laboratory animals. In addition, special attention should be paid to microbiological quality control of genetically engineered animals because the genetic alteration of the animals may result in modifications of the immunological competence. There are also infections which appear to be peculiar to nude mice. Therefore, a rigorous microbiological control is an essential part of developing of laboratory animal models.

According to the invention described herein, animals i.e. mice are colonized to have a refined intestinal bacterial flora, and reared under a strict barrier system. If animals are received from other institutions, where animals are kept in conventional facilities without strictly controlled microbiological monitoring, the animals must be cleaned by using the Alternative Microbiological Quality Control Method (AMQCM), which is an integral part of the present invention. By application of the AMQCM, the cost and time of microbiological control during developmental stages of animal models can be significantly reduced. The two-layer monitoring is performed to assure this microbiological quality.

Accordingly, this invention provides a new method for planned production of genetically engineered animals with strictly assured microbiological quality of their intestinal bacterial flora. The steps of in vitro fertilization (IVF), embryo cryopreservation, embryo transfer, nursing with recipient and/or foster mother are all integrated into this process.

In particular, eggs and sperms derived from animals suspected to have microbial infections are subjected to IVF to obtain aseptic embryos. The embryos are transferred into the uterus of recipient mice. Pups derived from IVF-embryos with infected mice sperms and eggs are microbiologically clean. Recipient and foster mother mice are supplied from strictly controlled mice stocks colonized by the refined intestinal bacterial flora (AC stock), so that pups possess the same flora during suckling period.

In the Alternative Microbiological Quality Control method, a modern, newly designed embryo banking facility is used in addition to ordinal barrier animal rearing system such as vinyl isolator. The embryo bank facility consists of three units 1) Quarantine Unit (QU), 2) Embryo Manipulation and Freezing Unit (EMFU), and 3) Recovery Unit (RU). An example of the embryo banking facility is shown in FIG. 13. The QU consists of an animal room to mooring microbiologically not assured donor, and aseptic equipment for collection of gamates (egg and sperm). The EMFU consists of an animal room for microbiologically clean donors, aseptic facility for gamate collection, aseptic IVF and freezing facility, and a room for liquid nitrogen tanks. The RU consists of rooms for recipient, embryo transfer, nursing and rearing. The EMFU and RU are equipped with a barrier system (filtered positive air condition, autoclave, clothes changing room, etc.) separated by the QU. Vinyl isolator or negative pressured animal rearing equipment is used in the QU with filtered positive air condition. Sterile locks are equipped between each room of RU, and between outside of barrier area and each room of the RU convenient for transfer of recipient and foster mother, embryos, etc. Pass boxes are equipped between facilities for gamate collection and IVF convenient for transfer of aseptic tubes.

In addition to these facilities, an isolator system is equipped for germ-free and gnotobiote animals colonized by refined intestinal bacterial flora (AC stock). These animals are carried into RU by vinyl isolator system through a sterile lock.

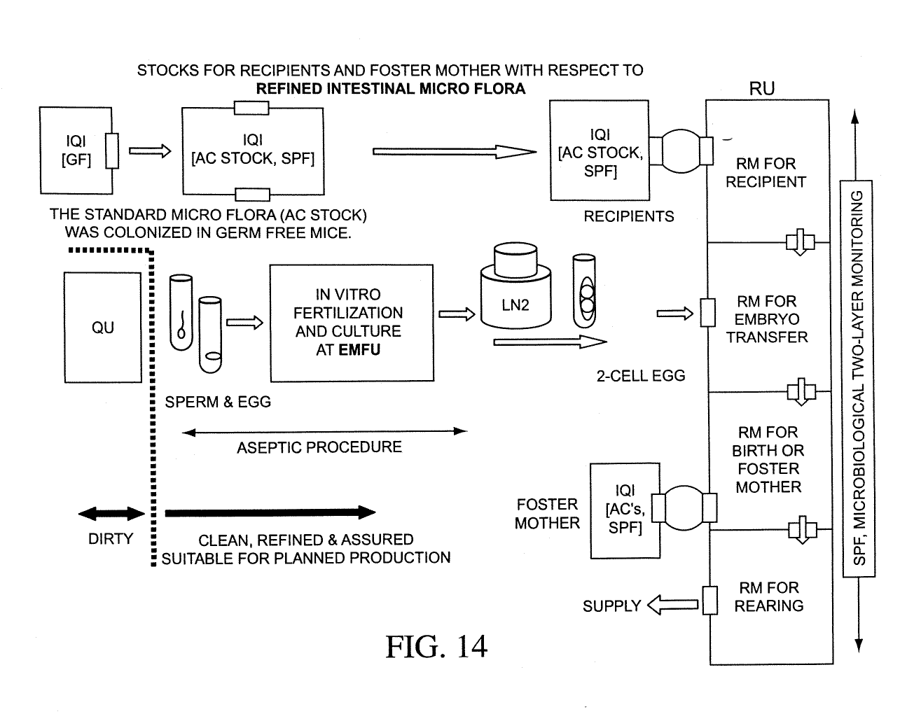

The Alternative Microbiological Quality Control Method is illustrated in FIG. 14. Animals i.e. mice suspected of outbreak or microbiologically not assured are accommodated in QU, while microbiologically assured animals i.e. SPF (Specific Pathogen Fee) are kept in EMFU. Donor mice are sacrificed and the surface of mice is sterilized. Eggs and sperms are collected aseptically, and transferred into the IVF facility through pass boxes. IVF is performed aseptically and cultured to two-cell embryos. The embryos are frozen in liquid nitrogen, or directly used for transplantation. The two-cell embryos are transferred into the RU through sterile lock, and transplantation is performed aseptically into recipient mice.

Pups delivered are naturally nursed by the recipient mother. In some case, pups delivered by Cesarean section are nursed by foster mother. In both cases, the intestinal bacterial flora (AC stock) is colonized into the intestine of the pups during nursing.

This method is further illustrated in Example 2.

Genetic Quality Testing

To use a mutant, such as genetically engineered animal, e.g. mouse, as an animal model, large numbers of genetically homogenous animals must be produced. Two types of genetic quality testing, depending on the aim, are illustrated in FIG. 4. If the aim is to clarify the genetic characteristics of the genetically engineered animals, spot checks are performed in order to determine in more detail the genetic characteristics of the given strain at a particular time. On the other hand, the assurance of consistent genetic quality requires monitoring of the animals, including periodic testing of a predetermined set of quality standards in order to confirm that there have been no changes in quality.

In most of the literature, molecular analysis of the transgene and/or its integration sites usually covers no more than five generations. The stability of germ-line transmission of the integrated transgene into the mouse host genome has not been the subject of detailed study. Reported observations about the transgene integration and stability are contradictory, and appear to be gene specific. It is evident that extensive molecular analyses of the integrated transgene are necessary not only to confirm stable integration, but also to eliminate or protect against genetic instability.

The genotype of a genetically altered animal (homozygote or hemizygote for transgenics) often differs from the genotype of the breeder pair for the same strain. The present invention provides a new method enabling not only monitoring of transgene stability in different generations, but also the genomic structure around the transgene integration site. The “early gene detection method” of the invention enables the differentiation between homozygous and hemizygous transgenic animals, e.g. mice, usually within two days. This is in contrast to the traditional approach, relying on sibling mating, which usually takes more than one month.

The examination of genetic stability of a transgene in any generation typically starts with determining the chromosomal localization, for example using the fluorescence in situ hybridization (FISH) method. (Matsuda et al., Cytogenet. Cell. Genet. 61: 282-285 (1992); Evans E P. Standard G-banded karyotype, In: Lyon M F, Rastan S., Bround S D M, eds., Genetic Variations and Stains of the Laboratory Mouse. New York: Oxford University Press; 1996, p. 1446-1449.) This is typically followed by Southern blot analysis, to prepare a restriction fragment map around the integrated transgene locus, which provides important information about the transgene architecture. The expression of the transgene can be confirmed by Northern blot analysis. Finally, the analysis is completed by reverse transcription PCR (RT-PCR) direct sequencing of the inserted gene and surrounding sequences, e.g. to identify point mutations that might occur in subsequent generations of the transgenic animals.

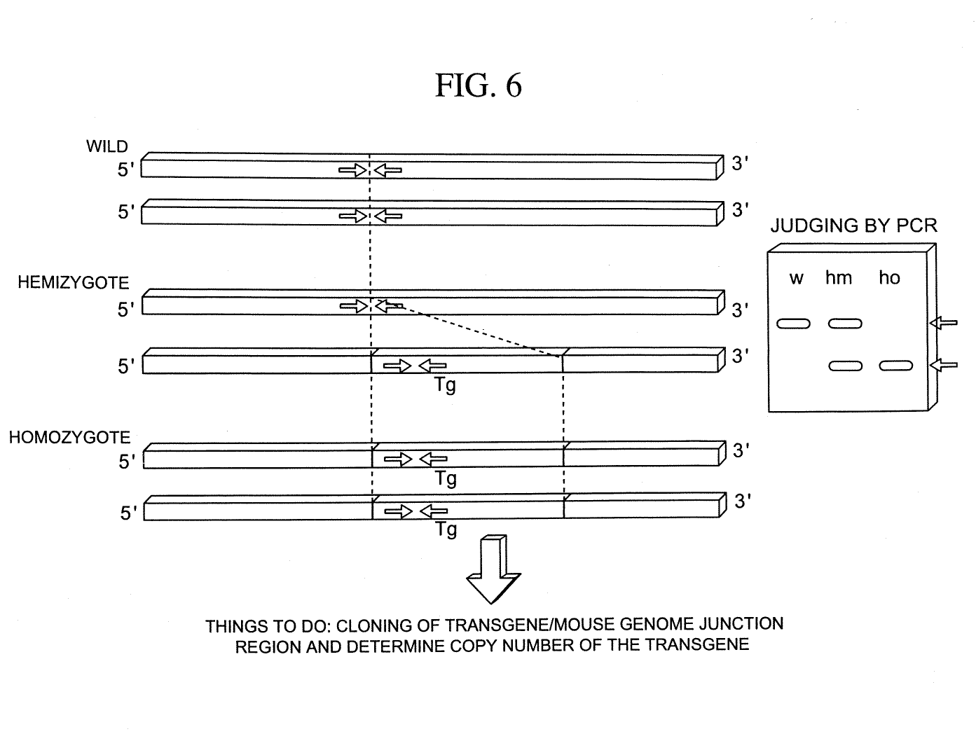

According to the present invention, transgenic animals are genotyped by using a new and efficient PCR approach. Gene specific PCR primers are designed to bind, in opposite directions, to complementary strands of the target. DNA isolated from the transgenic animal and the corresponding non-transgenic (wild-type) animal. Specifically, as illustrated in FIG. 6, PCR is performed with genomic DNA isolated from transgenic and non-transgenic animals, using the following primer pairs: (1) chromosome specific primer A and transgene specific primer C, for verification of the 5′ transgene/genome junction; (2) transgene specific primer D and chromosome specific primer B, for verification of the 3′transgene/genome j unction; (3) chromosome specific primers A and B, for identification of pre-integration site; and (4) transgene specific primers C and D, for verification of transgene-transgene junctions. The amplified PCR products created by using these primer pairs can be separated by size, for example on agarose gel, providing band patters that allow the identification of the genotype of any particular animal. Thus, hemizygotes will produce two bands, one corresponding to the wild-type allele, the oilier to the transgene. In contrast, homozygotes will show a band corresponding to the transgene only. In addition, differences in the PCR product resultant from the chromosome specific primer pairs A and B will reveal differences in the genetic background of animals.

Alternatively or in addition, the PCR products can also be separated or distinguished by signal differentiation, such as differentiation based on the color of the products labeled with fluorescence dyes. For example, when the transgene specific primer is labeled with FITC fluorescence dye, and the chromosome specific primer with HEX dye, the products from the transgene specific primer will exhibit a greenish color in contrast with the reddish color of the chromosome specific products, and can be distinguished based upon this property, using a fluorescence imaging detector. In this particular example, the products derived from DNA of hemizygotes are detected with yellowish color, resulting from a combination of green and red.

### Step 2: Planned Production and Supply System

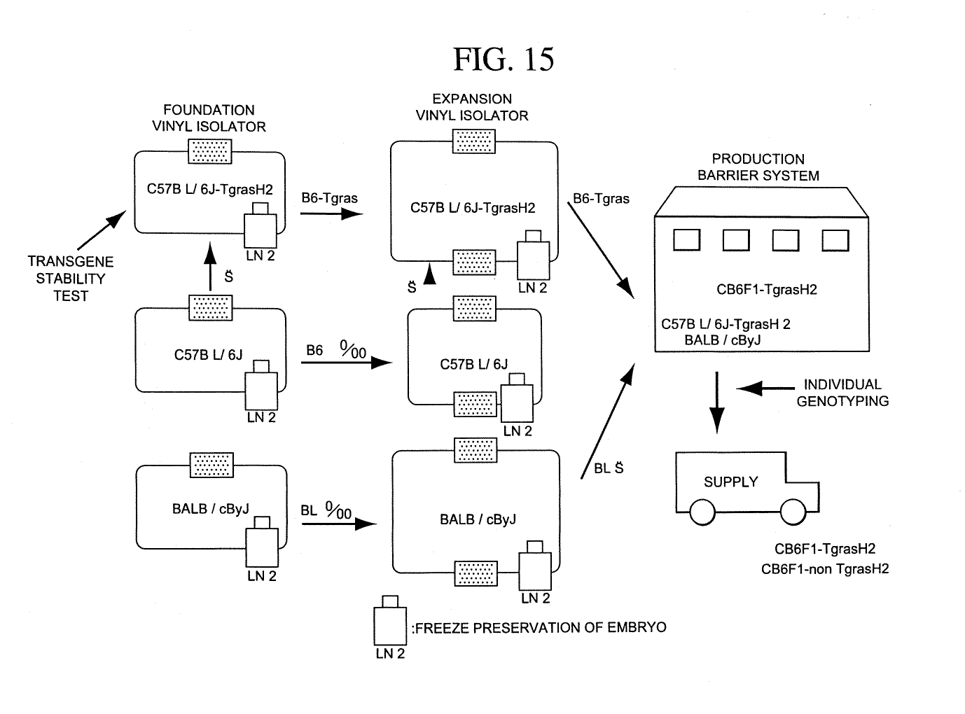

In addition, the invention provides for a planned production and supply system, which Includes (1) ongoing monitoring of transgene stability and function, (2) a risk management system (bulk preservation), (3) reproductive engineering technology, and (4) widening of the genetic background in order to achieve widened genetic diversity (see FIG. 15). The planned production is performed following the above four steps. The process uses nuclear, expansion, and production colonies to achieve step by step production, with freeze preservation of embryos. Using colonies is important for risk management. The step by step expansion of production is necessary to provide sufficient numbers of experimental animals (production lines) to evaluate their usefulness and limitations for the designated target human disease or physiologic function.

At the nucleus colony stage, the sib-mating fertilized eggs are preserved by cryopreservation. At the expansion and/or production stage, the eggs, after in vitro fertilization, are preserved as bulk by cryopreservation. The eggs are gathered from multiple female mice, while the sperm is gathered from a single male mouse in the bulk preservation. It usually takes tens of months to establish a production colony by natural impregnation. The cryopreservation system for its pedigree line in the nuclear colony, as well as the bulk preservation system in the expansion and production colonies, reduce the risk of accidents, such as contamination in the planned production, or other problems which lead to the discontinuance of production.

The establishment of nuclear colony and the determination of the genotype for the animals by the planned production and supply system is accomplished within a much shorter time period than usual when the novel method of this invention is applied. Indeed, transgene stability and genotype are checked within a day in every generation. Accordingly, the present invention enables the quick supply of experimental animals of any desired weight or age according to the user's specifications. This is a significant improvement over the conventional procedure, where supply of infant animals has been very difficult.

### Step 3: Evaluation of the Usefulness and Limitations of the Animal Model

For an animal model of a human disease to be truly useful, it must be defined, and only animals that meet the following requirements can be considered as defined animals models: the physiologic or pathologic phenotype which resembles that in humans must have a genetic cause identical to that in humans, and the usefulness and limitations of the animals a models must be defined. These requirements apply equally to all genetically engineered animals to be used as animal models of human diseases.

An example underlying the importance of this step is an animal model which has been developed in Japan for modeling Duchénne-type muscular dystrophy. At the start of the project, almost all the animals used internationally as models for muscular dystrophy were collected and provided to a clinical research group for evaluation (see, e.g., Gordon et al., PNAS USA 77: 7350-7384 (1980); Sugita and Nonaka, Animal models utilized in research on muscular diseases in Japan, p. 271-286 in: J. Kawamata and E. C. MeI by (ed.), Animal models: assessing the scope of their use in biomedical research. Alan R. Liss, Inc., New York; Bulfield et al., PNAS USA 81: 1189-1192 (1984); Tanabe et al., Acta Neuropathol 69: 91-95 (1986)). These muscular dystrophy models from spontaneous mutants were very useful in clarification of the pathogenesis of the disease, but were of little use in the study of the Duchénne-type muscular dystrophy. As the research progressed, it was found that these models had a disease with only signs resembling those of human muscular dystrophy. Similar considerations apply in the evaluation of the usefulness of all genetically engineered animal models, including transgenic, know-out and knock-in animals. For further details see, for example, Nomura, Laboratory Animal Science 47: 113-117 (1997).

An animal for which the usefulness and limitations in the elucidation of a mechanism of human disease have been evaluated is defined as an animal model for human disease. To take transgenic mice carrying the poliovirus receptor gene (TgPVR mice) as an example, the susceptibility of the TgPVR mouse to neurovirulence of the poliovirus is compared with the poliovirus neurovirulence of the human disease polio, in order to determine if they are the same. The animal for which usefulness and limitations in the elucidation of the target disease (e.g. susceptibility to poliovirus neurovirulence/polio) are evaluated is defined as a human disease 1 model useful for elucidation of that disease.

Further details of the invention are illustrated by the following non-limiting examples. Example 1 is an illustration of the super speed congenic method. Example 2 illustrates the Alternative Microbiological Quality Control Method (AMQCM) of the invention. Examples 3 and 4 illustrate the determination of transgene stability in Tg-rasH2 and TgPVR21 transgenic mice, respectively. Example 5 describes the new approach of the invention for the analysis of the transgene/mouse genome junction site. Example 6 illustrates the approach of the invention for widening the genetic background of transgenic animals in order to achieve widened genetic diversity. Examples 7-9 are provided as validations of the technology of the present invention through testing different transgenic animal models.

### Example 1

### Super Speed Congenic Method

The super speed congenic method is graphically illustrated in FIG. 4. It has been found that sexually premature young mice (immature mice) are sensitive to exogenous gonadotropin. Accordingly, superovulation in such immature mice can be induced by injections of the gonadotropin. The use the superovulation procedure for animal production significantly shortens the period required for changing the genetic background of the mutant mice, such as transgenic (Tg) mice, to that of other inbred strains, compared to the traditional procedure based on natural mating. In this study, the suitable conditions to induce superovulation and the developmental ability of the ovulated oocytes after in vitro fertilization in immature mice were examined.

### Materials and Methods

Immature C57BL/6N female mice (3 to 4 weeks of age) were subjected to superovulation procedure and mature males of the same strain were used as sperm donors for in vitro fertilization (IVF).

The immature female mice at 23, 24, 25, 26, and 27 days of age were induced to superovulate, using 1.25, 2.5, 5, 10, or 20 IU pregnant mere serum gonadotrophin (PMSG) and 5 IU human chorionic gonadotorophin (hCG), respectively, injected 48 h apart. After 17 to 20 h post hCG, the number of ovulated oocytes were assessed in each group. Some oocytes derived from 28-day-old mice were subjected to IVF procedure and cultured in vitro to the 2-cell stage. The obtained 2-cell stage embryos were transferred to the oviducts of mature Jcl: MCH (ICR) female mice on day 1 of pseudopregnancy to evaluate their fetal development.

### Results and Discussion

The proportion of mice that were induced ovulation and the number of ovulated oocytes were not related to the age of the examined mice. When 1.25 to 10 IU PMSG was injected, 75 to 100% mice were induced ovulation. The maximum number of ovulated oocytes was obtained by injection of 5 IU PMSG in each group (means 52.3 to 76.3 oocytes per mouse). The effect of inducing ovulation by injection of 15 or 20 IU PMSG was less than that of the other doses.

To assess normality of ovulated oocytes, the oocytes derived from 28-day-old mice were subjected to IVF procedure. The result showed that more than 95% of the oocytes were fertilized and developed to the 2-cell stage. After embryo transfer to recipient mice, more than 50% of the obtained embryos yielded to offspring, suggesting that the oocytes derived from immature mice have normal developmental activity.

These results demonstrated that normal oocytes could be obtained from about 4-week-old immature mice by injections of PMSG and hCG, in which the number of oocytes from immature mice was three to four times that from mature mice, and the oocytes possessed the ability toward normal fetal development. Using this procedure, back-crossing of the Tg mice with other strains was started to establish new congenic mouse strains. Using immature mice, as described above, backcrossing can be performed once every 48 days.

### Example 2

### Alternative Microbiological Quality Control Method (AMQCM)

### Materials and Methods

Mice: C57BL/6N mice (10 weeks of age) were used for virus infection; JCL:MCH (ICR) mice (10 to 15 weeks of age) were used as recipients. Virus: Sendai virus (HVJ MN strain) and mouse hepatitis virus (MHV Nu-67 strain) were used. Serological examination: Enzyme linked immunosorbent assay (ELISA) and hemagglutination inhibition (HI) test were performed for HVJ, while ELISA and complement fixation (CF) test were performed for MHV. The virus was infected to C57BL/6N mice through the nose of mice (day 0, the day of experiment start). PMSG (day 2) and hCG (day 4) were injected into virus-infected C57BL/6N mice for ovulation. Eggs and sperms were collected from the infected mice on day 5 for in vitro fertilization (IVF), and two-cell embryos were transferred into the oviducts of JCL:MCH (ICR) mice. After parturition (on day 25), pups were nursed by foster mother until weaning (day 53). Weaned mice were reared till day 81, and subjected to serological examinations. Serological examinations were also performed in recipient mice and virus-infected donor mice.

### Results

IVF and embryo transfer: In total, 60 eggs were collected from five HVJ-infected C57BL/6N mice (average: 12.0 eggs/mouse), and 44 eggs (73.3%) were developed to 2-cell egg. Forty 2-cell embryos were transferred into recipients and 22 pups (55.0%) were born, finally, 20 mice (90.9%)) were weaned. On the other hand, in total, 163 eggs were collected from five MHV-infected C57BL/6N mice (average: 16.3 eggs/mouse), and 145 eggs (89.0%) were developed to 2-cell egg. Eighty 2-cell embryos were transferred into recipients and 45 pups (56.3%) were born, finally, 39 mice (86.7%) were weaned.

Virus detection: HVJ-infected donor mice (one male and 5 female) were subjected to ELISA and HI test. All of samples tested were showed over-scaled in optical density in ELISA; over 1:160 titer in CF test (range 1:320 to 160). MHV-infected donor mice (one male and 5 female) were subjected to ELISA and CF test. All of samples tested were showed over-scaled in optical density in ELISA; over 1:20 titer in CF test (range 1:160 to 20). These results indicate that the virus was exactly infected in donor mice. While, recipient mice transferred embryos derived from HVJ-infected donor and from MHV-infected donor (each 3) were subjected to serological examinations. In addition, pups derived from HVJ-infected donor sperm and egg and from MHV-infected (each 5) were subjected to serological examinations. No recipient mice and pups tested showed positive test results in any serological examination.

### Example 3

### Transgene Stability and Features of rasH2 Mice as an Animal Model for Short-Term Carcinogenicity Testing

### Materials and Methods

### Animals

The transgene was constructed by ligation of each normal part of human activated c-Ha-ras genes with single point mutation at the 12th codon or the 61st codon, and then subcloned into the BamHI site of pSV2-gpt plasmid (Sekiya T, et al., Proc Natl Acad Sci USA 1984; 81: 4771-4775; SekiyaT et al, Jpn J Cancer Res 1985; 76: 851-855). The production of transgenic mice used in this study was described previously (Saitoh et al., Oncogene 1990; 5: 1195-1200). To maintain the foundation colony of the transgenic mouse, C57BL/6JJic-TgN (RASH2) (Tg-rasH2) mice were obtained by backcrossing male hemizygous rasH2 transgenic mice to female inbred C57BL/6JJic mice. In this study, 5 week old male Tg-rasH2 mice naturally mated with N20 and Tg-rasH2 mice al N15 obtained from cryopreserved embryos, and 12 week old male C57BL/6JJic (non-transgenic) mice were used. All animals used were handled in accordance with the guidelines established by the Central Institute for Experimental Animals, Japan.

### DNA Probes

An aliquot of microinjected DNA (7.0-kb BaniHI fragment) was subcloned into the BamHI site of pBlueScript II KS+ (pBSII: Stratagene, La Jolla, Calif.) plasmid. The plasmid was purified by CsCI equilibrium centrifugation followed by gel filtration on a Sepharose CL6B column (Amersham Pharmacia Biotech Inc., Buckinghamshire, UK). The 7.0-kb Bam HI fragment including the human c-Ha-ras gene was excised from the plasmid by Bam HI digestion and recovered from agarose gel. The 7.0-kb Bam HI fragment was labeled with digoxigenin (DIG)-11-dUTP using the DIG DNA labeling kit (Roche Diagnostics GmbH, Mannheim, Germany) according to the manufacturer's instructions (DIG-labeled random primed probe). The DIG-labeled 5′-probe (from the 1,793 to 2,400 position) was prepared by the PCR DIG Probe Synthesis Kit (Roche Diagnostics GmbH) using the 7.0-kb Bam HI fragment as template DNA with the following primers (forward; 5′-CCGACCTGTTCTGGAGGACGGTAACCTCAG-3′ (SEQ ID NO: 1), and reverse; 5′-ACCAGGGGCTGCAGCCAGCCCTATC-3′ (SEQ ID NO: 2)).

### Fluorescence In Situ Hybridization Analysis

Chromosomal location of the transgene was determined using the fluorescence in situ hybridization (FISH) method (Matsuda et al., Cytogenet Cell Genet, supra; Evans E P, supra). Twenty metaphases derived from mitogen-activated splenocytes obtained from Tg-rasH2 mice at N15 and N20 were analyzed with the biotin-16-dUTP-labeled 7.0-kb Bam HI fragment of the human c-Ha-ras gene. The biotin-labeled DNA was visualized with an anti-biotin goat antibody (Vector Laboratories Inc. Burlingame, Calif.) and a fluorescein isothiocyanate labeled anti-goat immunoglobulin G (Nordic Immunological Laboratories, Capistrano Beach, Calif.) and then counterstained with propidium iodide (Sigma-Aldrich Chemie GmbH, Steinheim, Germany). Observations were carried out with MICROPHOTO-FXA (NIKON CORPORATION, Tokyo, Japan) and chromosomes with fluorescent signals were identified according to the G banding standards (Evans E P, supra).

### Southern Blot Analysis

Genomic DNA was prepared from tail biopsies of Tg-rasH2 mice and non-transgenic mice by overnight incubation with proteinase K and subsequent extraction with phenol: chloroform and ethanol precipitation according to the standard protocol (Sambrook J, Russell D W. Molecular Cloning, Third Edition: A Laboratory Manual. New York: Cold Spring Harbor Laboratory Press; 2001). Genomic DNA, typically 10 μg, was digested overnight at 37° C. with 3 U of restriction enzyme per microgram of DNA, and ethanol precipitated at −20° C. After precipitation, the genomic DNA samples were resolubilized in 10 μl of TE buffer (10 mM Tris, pH 7.5, 1 mM EDTA) and electrophoresed overnight on 0.6% agarose gel. The gel was sequentially depurinated in 75 mM HCl, denatured in 1.5 M NaCl/0.5 M NaOH, and neutralized in 1.5 M NaCl/0.5 VI Tris-HCl, pH 7.5. The DNA was transferred from the gel to a nylon membrane (Hybond-N+, Amersham Pharmacia Biotech Inc.) overnight by capillary transfer in 25 mM sodium phosphate buffer, pH 7.0. The membrane was air dried and ultraviolet cross-linked. After a brief rinse in 2× standard saline citrate (SSC; 0.3 M NaCl and 30 mM Trisodium citrate, pH 7.0), the membrane was prehybridized for 6 hr at 42° C. in Church hybridization buffer (Church G M, et al., Proc Natl Acad Sci USA 1984; 81: 1991-1995) in a hybridization oven. The probe was denatured by boiling for 5 min and added to the blot in the fresh Church hybridization solution. The blot was hybridized overnight at 42° C. and then washed twice with 2×SSC/0.1% sodium dodecyl sulfate at 50° C. and twice with 0.2×SSC/0.1% sodium dodecyl sulfate at 65° C. The hybridized probes were detected by the DIG Luminescent Detection Kit (Roche Diagnostics GmbH) according to the manufacturer's instructions. For detection of the chemiluminescent signals, the blot was exposed to ECL-Plus X-ray film (Amersham Pharmacia Biotech Inc.).

### Northern Blot Analysis

Total cellular RNA was extracted using TRIzol (Life Technologies Inc. Gaithersburg, Md.). The RNA solution was treated with DNase I (Life Technologies Inc.) according to the manufacturer's protocol. RNAs (10 μg) were fractionated on 1% agarose/6% formaldehyde gel and transferred onto a Hybond-N⁺ nylon membrane. The blot was air-dried, ultraviolet cross-linked and hybridized as described previously (Maruyama et al., Oncol Rep 2001; 8: 233-237). The 7.0-kb BamHI fragment of human c-Ha-ras gene and murine glyceraldehyde-3-phosphate dehydrogenase cDNA was labeled with [α-³²P]-dCTP by the Random Primed DNA Labeling Kit (Roche Diagnostics GmbH) and used as a hybridization probe. The membrane was exposed to Kodak AR film.

### Cloning of Genoane/Transgene Junction Regions

For cloning of genome/transgene junctions, 100 μg of genomic DNA from Tg-rasH2 mice was completely digested with the restriction enzymes Hindi plus BamHI, and then extracted with phenol: chloroform and precipitated by the standard procedure (Sambrook J, et al, supra.). Six to 9-kb fragments of double-digested DNA were fractionated by ultracentrifugation on sucrose density gradient and ligated to the same sites of pBSII plasmid. Polymerase chain reaction (PCR) was performed with vector-ligated genomic DNA as the template using a recombinant Taq DNA polymerase (TaKaRa Inc. Shiga, Japan) according to manufacturer's instructions. PCR primers, pBSII-rev (5′-GGAAACAGCTATGACCATGATTACGC-3′ (SEQ ID NO: 3)) and C (5′-GACCGGAGCCGAGCTCGGGGTTGCTCGAGG-3′ (SEQ ID NO: 4)) were used for amplification of the 5′genome/transgene junction; pBSII-rev and D (5′-ATCTCTGGACCTGCCTCTTGGTCATTACGG-3′ (SEQ ID NO: 5)) were used for amplification of the 3′ transgene/genome junction. The reaction mixtures were heated to 94° C. for 2 min then amplified for 35 cycles at 94° C. for 30 sec, at 66° C. for 30 sec and at 72° C. for 3 min, after which the mixture was kept at 72° C. for 5 min in a ABI PCR2400 (Applera Corporation, Applied Biosystems, Foster City, Calif.). Nucleotide sequences of Hindi adjacent regions were determined by an ABI PRISM 310 Genetic analyzer (Applera Corporation) using ABI PRISM BigDye Terminator Cycle Sequencing Ready Reaction Kits (Applera Corporation). To isolate the genome/transgene junctions, two new primers, A (5′-GGGTCCTCTGGAGCTGGAGTTACAGACTAC-3′ (SEQ ID NO: 6)) and B (5′-GCTTGGCTfAAGATACAGCAGCTATCCTG-3′ (SEQ ID NO: 7)) were designed based on the sequence determined by the PCR cloning method. PCR amplifications were carried out with Tg-rasH2 mice genomic DNA as a template and primers C plus A (for the 5′genome/transgene junction), and D plus B (3′ transgene/genome junction). For cloning of transgene/transgene junctions, PCR amplification was earned out with Tg-rasH2 mice DNA and primers D and C. To clarify the integration processes and the possible position effects caused by transgene insertion, the pre-integration site was amplified with primers A and B from non-transgenic and Tg-rasH2 mice DNA. PCR conditions were the same as described above.

Sequencing of the Integrated Human c-Ha-ras Gene

Five overlapping PCR products that cover the overall integrated human c-Ha-ras gene were obtained form Tg-rasH2 at N20 by PCR using primers D (see above) plus E (5′-CACGCACCCAAATTAGAAGCTGCTGGGTCG-3′ (SEQ ID NO: 8)), F (5′-CCGACCTGTTCTGGAGGACGGTAACCTCAG-3′) plus G (5′-CACACGGGAAGCTGGACTCTGGCCATCTCG-31 (SEQ ID NO: 9)), H (5′-AAACCCTGGCCAGACCTGGAGTTCAGGAGG-3′ (SEQ ID NO: 10)) plus I (5′-AACCTCCCCCTCCCAAAGGCTATGGAGAGC-3′ (SEQ ID NO: 11)), and J (5′-TGCGCGTGTGGCCTGGCATGAGGTATGTCG-3′ (SEQ ID NO: 12)) plus K (5′-GTGCTGGGCCCTGACCCCTCCACGTCTGTC-3′ (SEQ ID NO: 13)).

PCR products were purified using the UltraClean PCR Clean-up DNA Purification Kit (Mo Bio Laboratories Inc., Solana Beach, Calif.) and nucleotide sequences were then determined by the primer walking method.

### Results

### Examination of Transgene Stability in Tg-rasH2 Mice

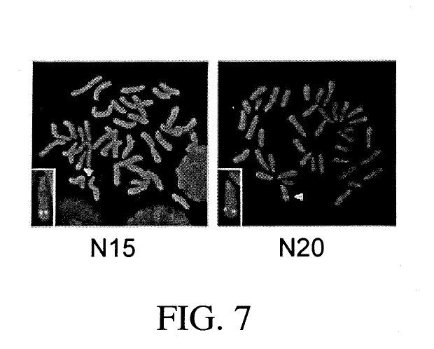

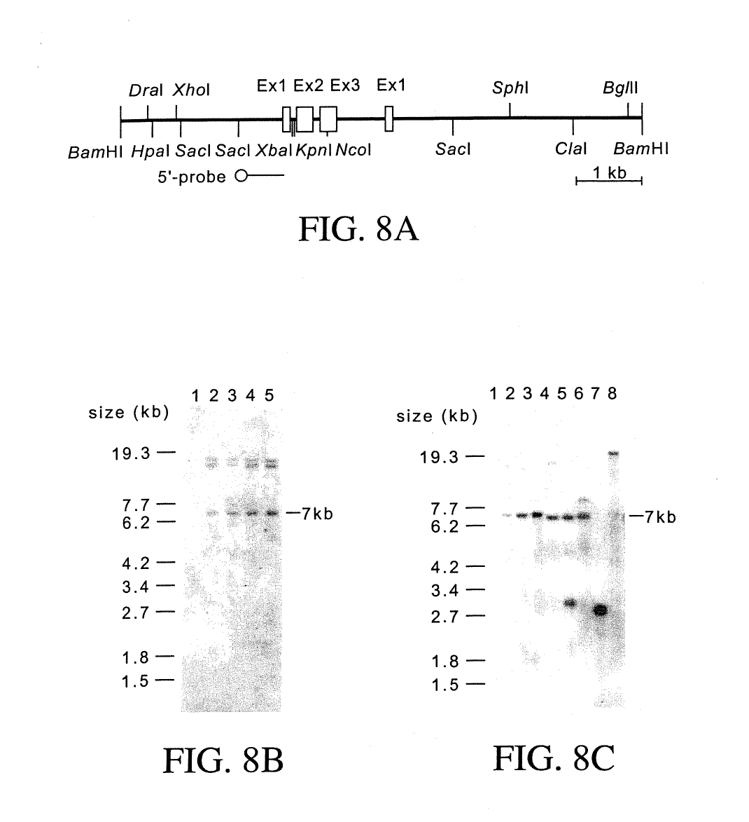

The transgenic mouse line rasH2 was established by Saitoh et al. in 1990, by microinjecting 7.0-kb of construct (BamHI fragment) containing human c-Ha-ras gene illustrated in FIG. 8A. The founder mouse was originally created as a hybrid (C57BL/6J x DBA/2J) strain and backcrossed to C57BL/6JJic, to make a genetically homogeneous population. Since, backcrossing has progressed beyond N20 and more than 30,000 transgenic mice have been produced. During large-scale propagation, through many generations, genetic stability of the integrated transgene in the Tg-rasH2 mice genome has been examined. Chromosomal localization of the integrated transgene in Tg-rasH2 mice at was determined at N15 and N20 by the FISH method. A paired fluorescent signal representing the transgene location was observed on the chromosome 15E3 region in both cases (FIG. 7). The Tg-rasH2 mouse is a hemizygote, so the hybridization signal was only detected in one pair of sister chromatids.

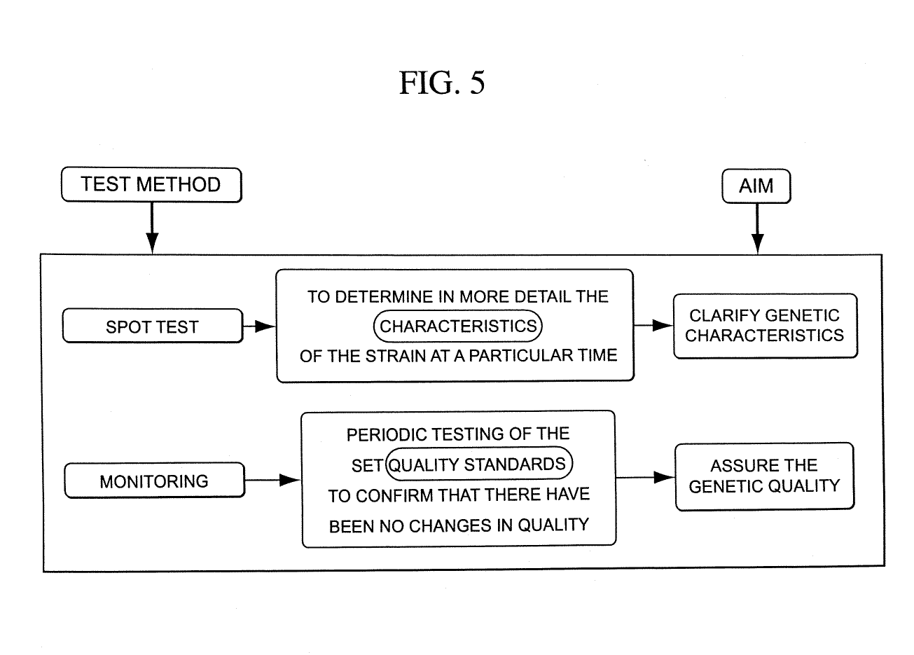

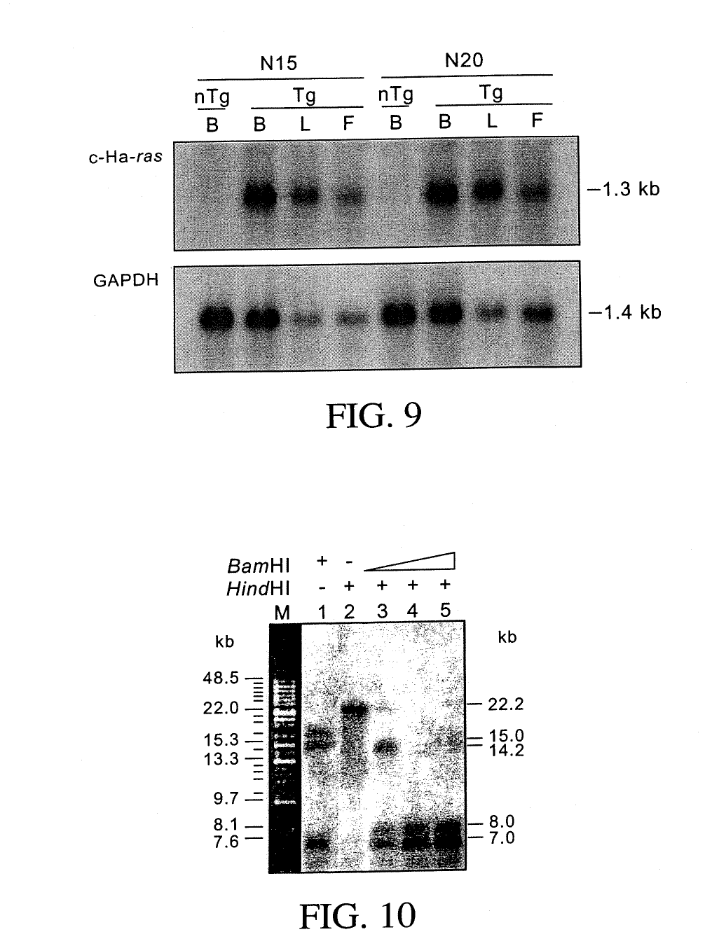

Southern blot analysis was carried out to prepare the restriction fragment map around the integrated transgene locus and it provided important information for transgene architecture. Digestion of Tg-rasH2 mice DNA with BamHI created three bands (7.0-kb and two higher molecular weight bands) hybridized with DIG-labeled random primed probe (FIG. 8B). No differences in the hybridizing band pattern were observed between Tg-rasH2 mice at N15 (FIG. 8B, lanes 2 and 3) and N20 (FIG. 5B, lanes 4 and 5). Digestion of non-transgenic mouse DNA with BamHI did not create any hybridizing band with the same probe (FIG. 5B, lane 1). The hybridization with DIG-labeled 5′-probe (FIG. 8C, lane 1) or DIG-labeled 3′-probe (positions from 6,024 to 6,712; data not shown) to BamHI-digested Tg-rasH2 mice DNA also showed the same hybridizing band pattern obtained by using DIG-labeled random primed probe. We confirmed expression of the transgene by Northern blot analysis. Expression of the human c-Ha-ras gene was observed in the Tg-rasH2 mice brain (B), but not in non-transgenic mice. In addition to the brain, the lung (L) and forestomach (F) expressed the transgene in each generation (N15 and N20) of Tg-rasH2 mice (FIG. 9). Reverse transcription PCR (PT-PCR) direct sequencing analysis revealed that point mutations that preferentially occurred at codon 12 and 61 in the human c-Ha-ras gene were not seen in either generation of Tg-rasH2 mice. Other than in the mutation hot spots, no nucleotide changes were seen in the coding region (data not shown).

Determination of Transgene Orientation and Copy Number inTg-rasH2 Mice

To clarify the integrated transgene architecture, Tg-rasH2 mice genomic DNA was digested with several restriction enzymes (HpaI, XhoI, XbaI, NcoI, NglII) that cut at a known single site in the transgene and was subjected to Southern blot analysis. If the integrated transgenes were present in tandem in the head-y-tail configuration, these restriction enzymes would produce a 7-kb fragment. XbaI digestion of direct repeating transgene copies would produce a 7-kb fragment, whereas an inverted repeat would produce a 9.1-kb (tail-to-tail) or a 4.9-kb (head-to-head) fragment. Actually, digestion of genomic DNA from a Tg-rasH2 mouse at N20 with XbaI produced a 7-kb hybridized band with DIG-labeled 5′-probe (FIG. 8C, lane 4). All other restriction enzymes that cut at a known single site in the transgene also created a 7-kb band hybridized with DIG-labeled 5′-probe (FIG. 8C, lanes 2, 3, 5 and 6). These results suggested that several copies of the integrated transgene were present in tandem in the head-to-tail configuration. The same hybridizing band pattern was also observed in Tg-rasH2 mice at N15 (data not shown).

To determine the copy number of the integrated transgene, Tg-rasH2 mouse DNA was digested completely with HindIII and then the aliquots were partially digested with various concentrations of BamHI restriction enzyme. The digested DNAs were electrophoresed on 0.4% agarose gel to resolve clearly high molecular weight DNA samples. Southern blot analysis with DIG-labeled random primed probe is shown in FIG. 10. When genomic DNA was completely digested with HindIII, only a 22.2-kb band was hybridized with DIG-labeled random primed probe (FIG. 10, lane 2). The 22.2-kb fragment can contain maximum three copies of the 7.0-kb transgene. HindIII and BamHI double-digestion created 8-kb and multiple 7-kb fragments hybridized with a DIG-labeled random primed probe (FIG. 10, line 5). In addition to 22.2, 8, and 7-kb bands, 14.2 and 15-kb bands were hybridized with DIG-labeled random primed probe, when HindIII digested genomic DNA was further partially digested with BamHI (FIG. 10, lane 3). These results demonstrated that Tg-rasH2 mice include three copies of the transgene in their genome.

Cloning and Sequencing of Genome/Transgene Junctions and their Corresponding Pre-Integration Site

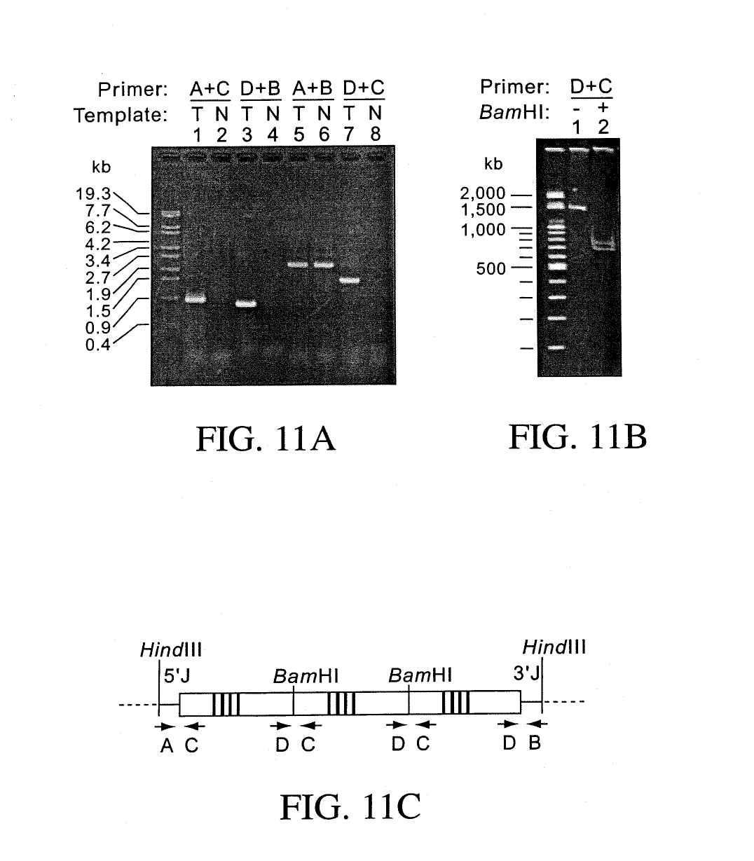

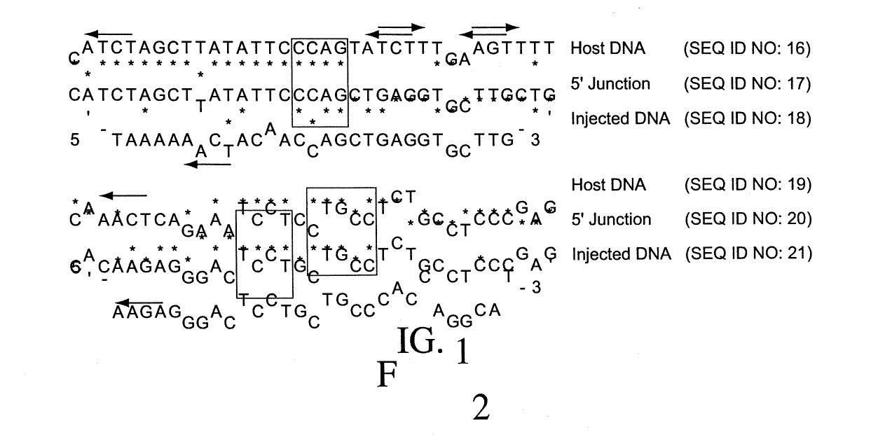

The results obtained from Southern blot analysis suggested that 7 and 8-kb fragments derived from Tg-rasH2 genomic DNA by Hind III and Bam HI double-digestion include genome/transgene and/or transgene/genome junction regions. To study fine structure of the genome/transgene junctions in the Tg-rasH2 mice genome, 6 to 9-kb of HindIII-BaniHI double digested fragments, which were fractionated by ultracentrifugation on sucrose density gradient, were ligated to the same sites of pBSII plasmid. Sequences positioned between two PCR primers were amplified by PCR (1st PCR) using the appropriate primers (pBSII-rev and C; for amplification of the 5′ genome/transgene junction, pBSII-rev and D; for amplification of the 3′ transgene/genome junction) and analyzed by PCR-direct sequencing (GenBank Accession No. AB072334). To eliminate the possibility that the amplified DNA fragments were an artifact of the PCR-cloning procedure, each side of the genome/transgene junctions was re-cloned from Tg-rasH2 mice genomic DNA by 2nd PCR with the following sets of primers (C plus A and D plus B, FIG. 11 C). Each of the PCR products amplified with primer set C plus A, and D plus B was only observed in Tg-rasH2 mice with a predicted size of 867-bp (FIG. 11A, lane 1) and 804-bp (FIG. 10A, lane 3), respectively. The nucleotide sequences of the 2nd PCR products coincided with the nucleotide sequences with 1st PCR products. These results suggested that the genome transgene junction sequences obtained by PCR-cloning actually exist in the Tg-rasH2 mice genome.

A PCR approach was employed to amplify and subsequently clone the pre-integration site from non-transgenic and Tg-rasH2 mice DNA. The pre-integration site was amplified using primers A and B within unique sequences flanking the site of insertion of the transgene. Primer set A plus B created a 2.2-kb PCR product in not only non-transgenic mice but also Tg-rasH2 mice (FIG. 11A, lanes 5 and 6). Furthermore, the 2.2-kb PCR product was also obtained from DBA/2J mice DNA (data not shown). In this experiment, we used the C57BL/6JJic mice as non-transgenic control to determine the pre-integration site. However, the original rasH2 mouse was generated in a C57BL/6J x DBA/2J hybrid strain, so we cannot exclude the possibility that the microinjected human c-Ha-ras gene was integrated into the DBA/2J allele. The Tg-rasH2 mouse is hemizygote and has one wild-type allele. The 2.2-kb fragment was found to contain mouse genomic DNA sequences, which may have been deleted in the Tg-rasH2 mice genome. We determined the nucleotide sequence of the 2.2-kb fragment by PCR-direct sequencing (GenBank Accession No. AB072335) and compared it with the sequences of the transgene/genome junctions (GenBank Accession No. AB072334) and micro injected DNA. DNA sequencing analysis revealed that a 1,820-bp sequence had been deleted when the microinjected human c-Ha-ras gene was integrated into the mouse host genome.

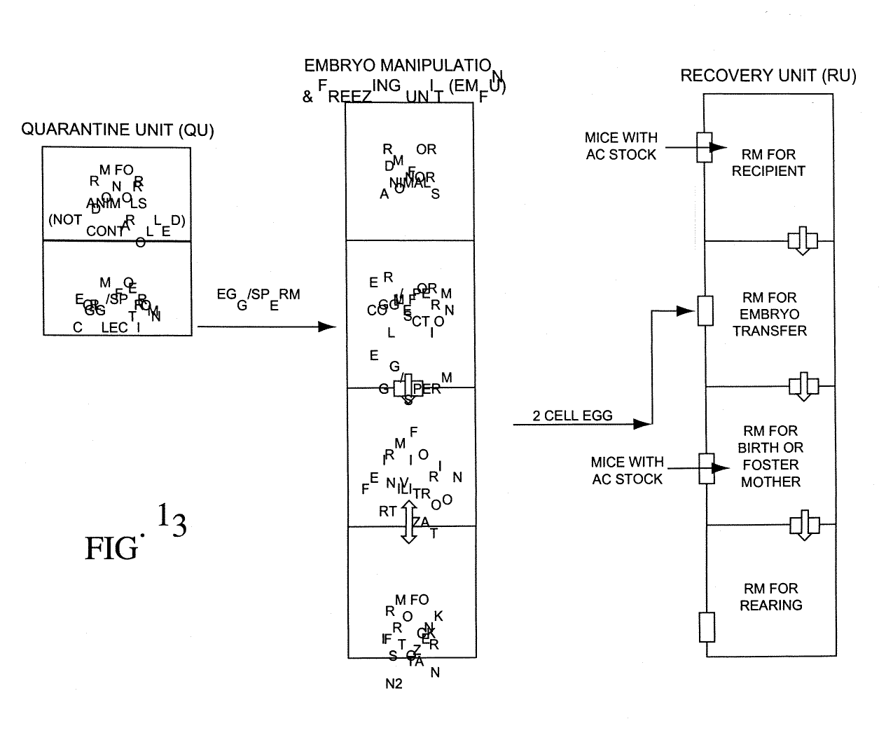

FIG. 12 compares the 5′ genome/transgene junction (5′J) and 3′ transgene/genome junction (3′J) sequences with the host genome and injected DNA sequences. A remarkable feature common to both the junctions was the presence of short homologies between the parental sequences. Spanning 5′J, there was a 148-bp deletion at the 5′end of the injected sequences, and a 4-bp homology (CCAG) between the parental sequences was present at the 5′ end of the final integrant. Spanning 3′J, there was 90% homology within a stretch where 10-bp (TCCTgCTGCC; the small letter indicating a mismatched position) was homologous between sequences at the 3′end of the transgene integrant, which had a 24-bp deletion at the 3′end and the parental sequences. Our results support the assumption that the short homologous pairings may have contributed to the chromosomal integration event. The consensus sequence for cleavage sites of mammalian topoisomerase I was found in the vicinity of 5′J and 3′J in the host genome. This sequence also appeared in the injected DNA near the 5′J and 3′J sites.

Sequence Analysis of the Transgenic Construct and the Integrated Human c-Ha-ras Gene in Tg-rasH2 Mice

Transgene/transgene junctions within the concatemer were analyzed by PCR-restriction fragment length polymorphism and PCR-direct sequencing. The PCR product amplified with primers D and C was only observed in Tg-rasH2 mice with the predicted size of 1.4-kb (FIG. 11A, lane 7, and 11B, lane 1). An amplified 1.4-kb fragment was divided into two fragments of 0.7-kb in size by BamHI digestion (FIG. 11B, lane 2). The PCR-direct sequencing also revealed that transgene/transgene junctions conserved the BamHI recognition sequence in the Tg-rasH2 mice genome and there have been no sequence losses or rearrangements at these junctions.

Sequence Analysis of the Transgenic Construct and the Integrated Human c-Ha-ras Gene in Tg-rasH2 Mice

The 7.0-kb construct was prepared by joining with each normal part of the c-Ha-ras gene derived from human melanoma and bladder carcinoma cell lines (Sekiya et al., PNAS USA 81: 4771-4775 (1984); Sekiya et al., Jpn Cancer Res 76: 851-855 (1985)). The nucleotide sequences of the c-Ha-ras gene in these cell lines have been registered on a public database (GenBank Accession No. M30539 and V00574). The 7.0-kb of the construct was a chimeric and artificial ras gene, so we did not know the precise nucleotide sequence of this construct used for microinjection. Therefore, we reconfirmed the nucleotide sequence of an aliquot of micro injected DNA. We determined the nucleotide sequence of the chimeric human c-Ha-ras gene (=7.0-kb of BainHI fragment, 6,992-bp). Several minor differences were seen in the chimeric human c-Ha-ras gene, when this sequence was compared with human c-Ha-ras gene sequences from melanoma and bladder carcinoma cell lines. However, we could not detect any changes in each exon. We also determined the nucleotide sequence of the integrated human c-Ha-ras gene. Five overlapping PCR products which cover the overall integrated human c-Ha-ras transgene were obtained by PCR using appropriate primers (see Materials and Methods) and analyzed by PCR-direct sequencing (GenBank Accession No. AB072334). We could not detect any differences between the nucleotide sequences obtained from the Tg-rasH2 mouse at N20 and the microinjected DNA except for small deletions at both ends of the tandemly arrayed transgene.

### Discussion

The original rasH2 mouse (a hybrid of C57BL/6J x DBA/2J) has been backcrossed to the C57BL/6JJic strain to create a genetically homogeneous population. At present, the backcrossing has progressed beyond N20. It appears that the genetic background of this transgenic line has been almost replaced with the C57BL/6JJic background (about 99.9998%, (Silver L M, Laboratory Mice. In: Silver L M ed., Mouse Genetics, Concepts and Applications, New York: Oxford University Press; 1995, p. 46-48). It is important to consider the genetic background of animals used in carcinogenicity testing because the spontaneous and chemically induced tumor incidences are different among mice strains. For short-term carcinogenicity testing, we have recommended the use of F1 hybrid rasH2 transgenic mice (CB6F1-Tg-rasH2) obtained by breeding female BALB/cByJ mice and male Tg-rasH2 mice. This unique breeding system has two advantages: one is that it is possible to achieve a wide variety of responses to chemical compounds, and the other is that it is possible to use sibling non-transgenic (CB6F1-NonTg) mice as the examination control.

In this study, we showed that the integrated human c-Ha-ras gene in Tg-rasH2 mice is stably transmitted over generations. DNA molecules microinjected into cultured cells or fertilized mouse eggs are usually integrated at a single site in the host genome and when these transgenes are present in multiple copies, they are arranged predominantly in head-to-tail tandem arrays and more rarely in head-to-head or tail-to-tail orientation (Filger et al., Mol Cell Biol 2: 1372-1387 (1982); Gordon and Ruddle, Gene 33: 121-136 (1985); Palmiter and Brinster, Annu Rev Genet 20: 465-499 (1986)).

Tg. AC transgenic mice are known to have a population not responsive to the positive control compound 12-0-tetradecanoylphorbol 13-acetate (Thompson et al., Toxicol Pathol 26: 548-555 (1998); Weaver et al., Toxicol Pathol 26: 532-540 (1998); Blanchard et al., Toxicol Pathol 26: 541-547 (1998)), and the nonresponder showed gene deletion near the apex of the head-to-head juncture of the inverted repeat (Thompson et al., Toxicol Pathol supra; Honchel et al. Mol Carcinog 30: 99-110 (2001). Several studies showed that the inverted repeat sequence with palindromic structure in transgenes caused instability of the gene (Akgun et al., Mol Cell Biol 17: 5559-5570 (1997); Collide et. al., EMBO J 15: 1163-1171 (1996); Ford and Fried, Cell 45: 425-430 (1986)). Fortunately, there are no palindromic structures in the tandemly arrayed human c-Ha-ras transgene in the Tg-rasH2 mice genome, but we do not know whether transgene rearrangement occurred during large-scale propagation over a large number of generations. Aigner et al. proposed that breeding programs could be continued to a high number of generations without further stringent molecular analysis in an established homozygous transgenic line by observing seven lines of tyrosinase gene transgenic mice (Aigner et al., supra). However, they noted that very few individuals were affected by a transgene copy loss in their experiment. We demonstrated here that the integrated transgene in Tg-rasH2 mice was stably transmitted over several generations and during large-scale propagation (FIGS. 7 and 8). In Southern blot analysis of 450 Tg-rasH2 mice, we did not find any differences among individual DNA samples (unpublished data). However, we believe that checking of the genotype and phenotype is required at regular intervals in Tg-rasH2 mice used for carcinogenicity testing because possible contamination with nonresponder mutant in the foundation colony will affect the reliability of carcinogenicity test results. Therefore, we should confirm the integrity of the parental Tg-rasH2 (C57BL/6JJic-TgN(RASH2)) mice at each generation by detailed molecular genetic analyses including Southern and Northern blots and PCR-direct sequencing of the expressed human c-Ha-ras gene (recent results at N23 with no obvious change, unpublished data). In addition, actual testing model CB6F1-Tg-rasH2 mice should be subjected to carcinogenicity testing with N-methyl-N-nitrosourea as a standard positive control compound.

Results of Southern blot analyses revealed the copy number of the Integrated transgene. Generally, it is difficult to determine the exact copy number of an integrated transgene because microinjected DNAs are reiterated to form tandem or inverted arrays ranging from about one to several hundred copies per site. In Tg-rasH2 mice, the microinjected human c-Ha-ras gene did not have any HindIII recognition site in its sequence. Therefore, the transgene integration locus was cut out of the Tg-rasH2 mouse genome by HindIII digestion and detected as a single 22.2-kb band by Southern blot analysis. If the intact 7-kb of human c-Ha-ras gene were integrated in the Tg-rasH2 mouse genome, the integrated transgene would not exceed three copies. In addition BamHI digestion created three bands hybridized with the random primed probe which would cover the overall of the 7-kb of human c-Ha-ras gene suggesting that the integrated transgene had a minimum of three copies. However, a similar banding pattern would be possible by integration of two copies of the gene if it had been present in a circular form. If so, BamHI digestion would create two hybridized bands when the hybridizations were carried out with 5′-probe covering positions 1,793 to 2,400 of 7-kb of the human c-Ha-ras gene (FIG. 8C, lane 1) or 3′-probe covering positions 6,024 to 6,712 (unpublished data). Both of the region specific probes hybridized and created three similar bands with those of the random primed probe. These results suggested that the integrated transgene had three copies. The existence of sequences for the genome/transgene junction at both ends (FIG. 12) also denies possible integration in the circular form.

Since it is not known if the transgene copies showed any deletion or rearrangement when the microinjected DNA was integrated into the mouse genome, we cloned the genome/transgene and the transgene/genome junctions from Tg-rasH2 mouse DNA, and their corresponding pre-integration sites from the non-transgenic mouse. It has been reported that the terminal sequences of the microinjected DNA were relatively conserved and modified by loss or insertion of a maximum of several nucleotides in transgenic mice (Pawlik et al, Gene 165: 173-181 (1995); Hamada et al., Gene 128: 1978-202 (1993); McFarlane and Wilson, Transgenic Res 5: 171-177 (1996)). From the results of Southern blot analysis, we suspected that both (5′ and 3′) ends of the tandemly arrayed transgene copies have some deletions in the Tg-rasH2 mouse genome. If the tandemly arrayed transgene was integrated intact, the integrated transgene copies would have conserved BamHI sites at their junctions and would create only the 7.0-kb monomeric fragment by BamHI digestion. Comparison of the sequences of the transgene/genome junctions and the microinjected DNA has revealed that Tg-rasH2 mice have a 148-bp deletion at the 5′end and a 24-bp deletion at the 3′end (GenBank Accession No. AB072334) on transgene integration. These deletions seen at both ends suggested that the transgene concatemers were present in a linear rather than a circular form until integration and that the free ends of the linear concatemers were the preferred sites for recombination. The nucleotide sequence analysis of the transgene integrated locus revealed the presence of short homologies (4-bp at 5′end and 9 out of 10-bp at 3′end) between the parental sequences at integration junctions. These short homologies between host genome and transgene at integration junctions have been observed in transfected fibroblasts and in transgenic mouse lines (Hamada et al., Gene 128: 197-202 (1993); McFarlane and Wilson, Transgenic Res 5: 171-177 (1996)). In addition. DNA topoisomerase I seems to play an important role in the integration of microinjected DNAs. The consensus sequence of the cleavage sites for mammalian topoisomerase I (Been and Burgess, Nucleic Acids Res 12: 3097-3114 (1984)) was found in the vicinity of integrated transgene sites in several transgenic lines (Hamada et al., supra. McFarlane and Wilson et al., supra) and the Tg-rasH2 mouse (FIG. 12).

It depends on cases of loss or rearrangement of host genome occurring on transgene insertion. In the host genome of Tg-rasH2, nucleotide deletion (1,820-bp) occurred when the microinjected human c-Ha-ras gene was integrated into the mouse host genome. The nucleotide sequence (GenBank Accession No. AB072335) deleted in Tg-rasH2 mice was compared with those from GenBank databases using the BLAST2 program to identify possible homologies. The deleted sequence did not have any homologies with known functional genes on the databases. However, the deleted region was found to carry a sequence homologous to human DNA sequence from clone RP6-1107 on chromosome 22 containing an RPL7 (60S Ribosomal Protein L7, GenBank Accession No. AL031589) pseudogene. The 312-bp of the deleted region sequence (position 698-1,009) showed 88% homology with the human DNA clone RP6-1107 (position 9,023-9,334), but sequence homology was not observed within the coding region of RPL7. Sequence homologies at the amino acid levels were not observed when the deleted sequence was translated with various frames and orientations into the corresponding amino acid sequences. Although tire possibility remains that the deleted sequence that we determined was located in an intron or a promoter region, insertion of the human e-Ha-ras gene into the host mice genome would not cause insertional mutation. The basal gene expression was not affected by the transgene insertion in Tg-rasH2 mice. This conclusion is supported by preliminary evidence from expression profiling approaches. We could not find marked differences between Tg-rasH2 mouse liver and non-transgenic mouse liver in comparison with the basal gene expression of 9,514 unigenes (unpublished data).

Tg-rasH2 transgenic mice, which are a genetically homogenous population and have been refined by molecular biological analyses including transgene architecture and alteration of the host genome sequence, should be a useful rodent model for short-term carcinogenicity testing.

### Example 4

### Transgene Stability of TgPVR21 Mice as an Animal Model for Neurovirulence Test (NVT)

A transgenic mouse which carries the human polio virus receptor (PVR) gene was created by Nomoto (PNAS, 88: 951-955.1991). The mouse has been developed as an animal model for the neurovirulense test (NVT), as an alternative to the monkey neurovirulence test (MNVT) at the Central Institute for Experimental Animals, Japan. Stability of the transgene is one of the essential factors to assure reproducible quality of the TgPVR21 transgenic mice as an animal model for NVT. To examine stability of the transgene in TgPVR21 mice, the molecular structure of the transgene was analyzed in different generations in a congenic process to the IQI strain.

Materials and Methods

Structure of the transgene in TgPVR21 mice was analyzed at backcross numbers N3, N15 and N20 to the IQI strain (Table A). FISH, Southern and Northern blot, and RT-PCR analyses were performed (Table A) following standard procedures, as described below. The nucleotide sequence of the coding region of the transgene was also determined.

Results

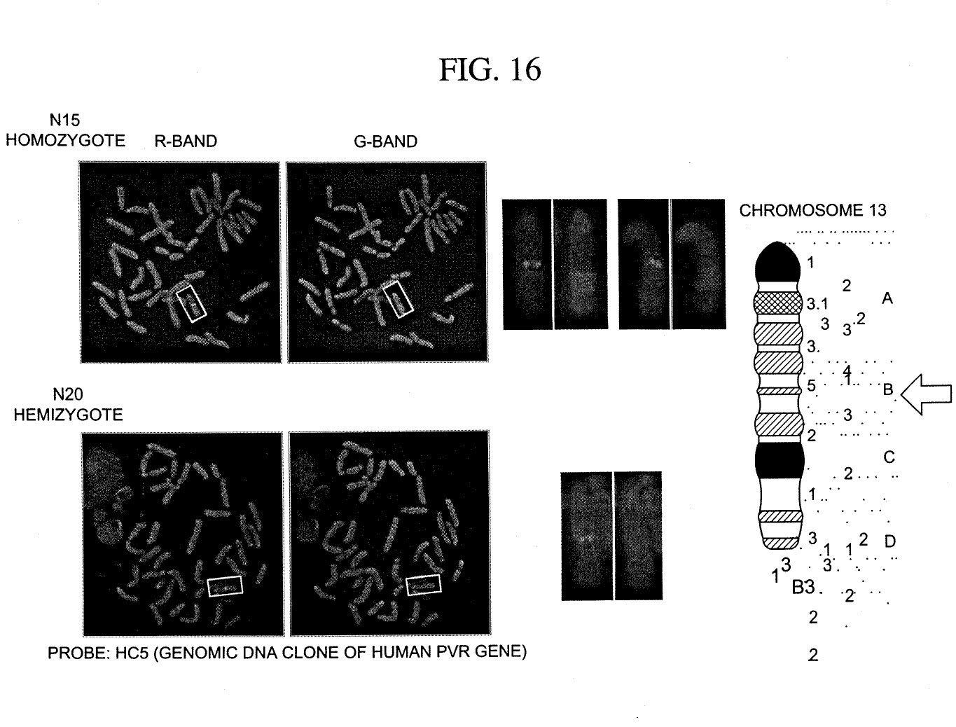

FISH (FIG. 16)

FISH analysis was performed using biotin-labeled HC5 clone as a probe and visualized by avidin-FITC method. As shown in FIG. 16, two twin spots and one twin spot were seen in chromosome No. 13 (position 13B3) of transgenic homozygote of N15 and hemizygote of N20, respectively. The chromosomal location of the transgene observed in this analysis was consistent with previous results (Nomoto, 1991, supra).

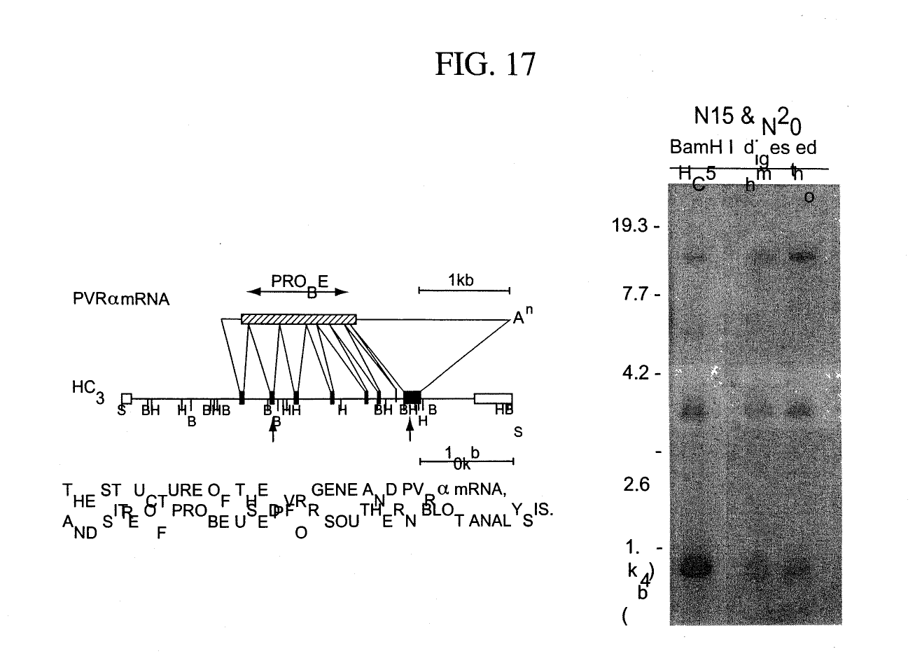

Southern Blot Analysis (FIG. 17).

Ten micrograms of DNA obtained from transgenic homozygote of N15 and hemizygote of N20 mice were digested with BamHI and subjected to agarose gel electrophoresis. DNA was transferred onto membrane. The membrane was hybridized with a probe shown in FIG. 17 (coding region of PVR-α). The hybridized bands ware seen at sizes of 1.2, 1.3 and 10 kb in both mice and control HC5 clone. These findings were consistent with previous results suggesting that no rearrangement occurred, and the transgene has been stable in the congenic process in the TgPVR21 strain.

Gene Expression Analyses.

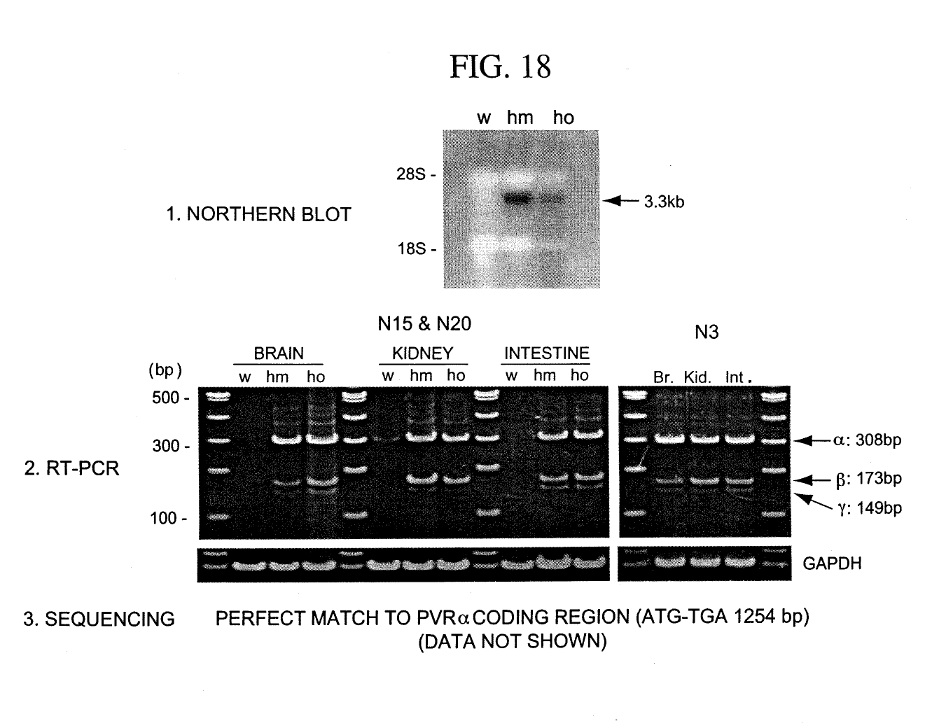

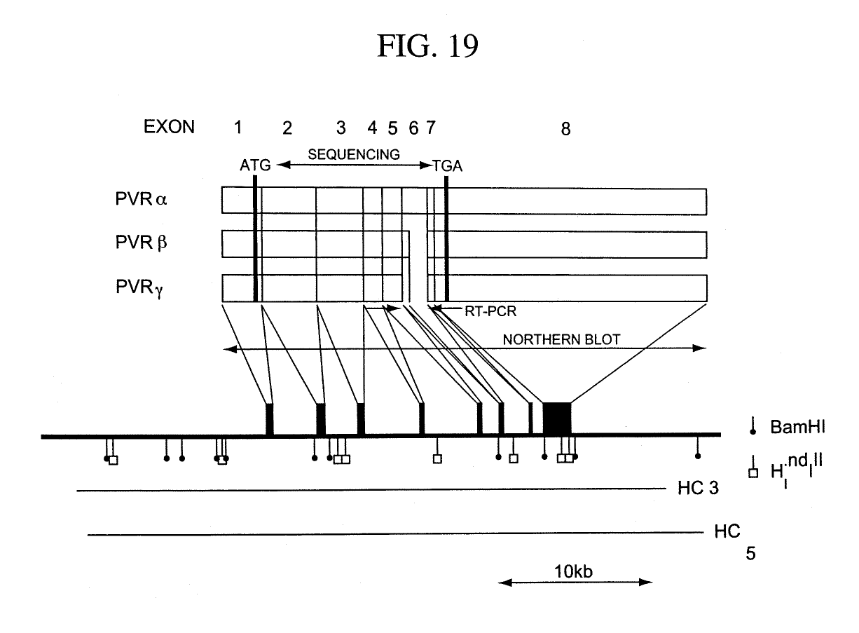

Northern blot, PT-PCR, and direct sequencing were performed to examine the gene expression profiles of TgPVR21 strain (FIG. 18). The structure of integrated transgene gene, and three mRNA products produced by gene splicing, probe for Northern analysis, primers and part of sequencing are shown in FIG. 19. Total cellular RNA was run in gel and transferred onto membrane. The membrane was hybridized with the probe shown in FIG. 17 (cDNA of PVR-α mRNA). A single 3.3 k band was detected in both N15 and N20 of TgPVR21 strain. The date obtained here was consisted with previous results (Nomoto, 1991, supra), RNAs obtained from brain, kidney and intestine of N3, N15 and N20 of TgPVR21 mice were subjected to PT-PCR analysis. Three types of RNA products (PVR-α, -β, and -γ) derived from the integrated PVR gene by alternative splicing were detected as expected size (149, 173 and 308 bp). PCR direct sequence method was performed using cDNA obtained from RNA of N15 mouse brain. The results confirmed that the integrated transgene produces RNA perfectly matched to the coding region of the PVR gene (1,254 bp, ATG as start codon to TGA as terminal codon).

### Example 5

### Analysis of Transgene/Mouse Genome Junction Site

It has been confirmed that following the production method of the present invention trangenes can be stably transmitted from generation to generation. This fact allows one to develop a novel method for genotyping the mouse. The integration site of the transgenene including the transgene/mouse genome junction region novel transgenic mouse strains (TgPVR21 mouse—see Example 4 above, and rasH2 mouse—Example 3 above) was cloned and analyzed in order to illustrated the novel genotyping method using these strains. The general concept of the novel genotyping method is illustrated in FIG. 6. In the Figure, darker arrows indicate PCR primers designed to detect wild type, and light arrows indicate PCR primers designed to detect the transgenic type of the mouse. By following this method, various genotypes, e.g. wild-type homozygote, hemizygote (or heterozygote), and transgenic homozygote can be easily and clearly distinguished. DNA was obtained from transgenic homozygote of TgPVR21 mouse.

Southern Blot Analysis

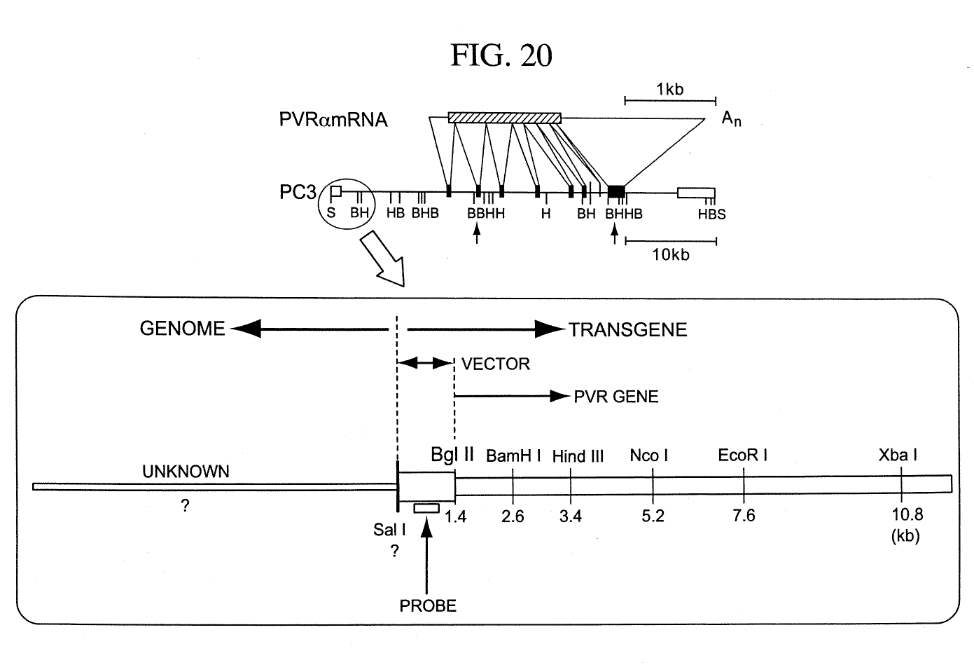

Southern blot analysis to obtain the restriction enzyme map for the 5 region of the transgene/mouse genome junction site was performed. BamHI, EcoRI, BglII, NcoI HindII, and XbaI were used for DNA digestion. A 700 bp segment of vector part of the transgene was used as probe for Southern blot analysis (FIG. 20).

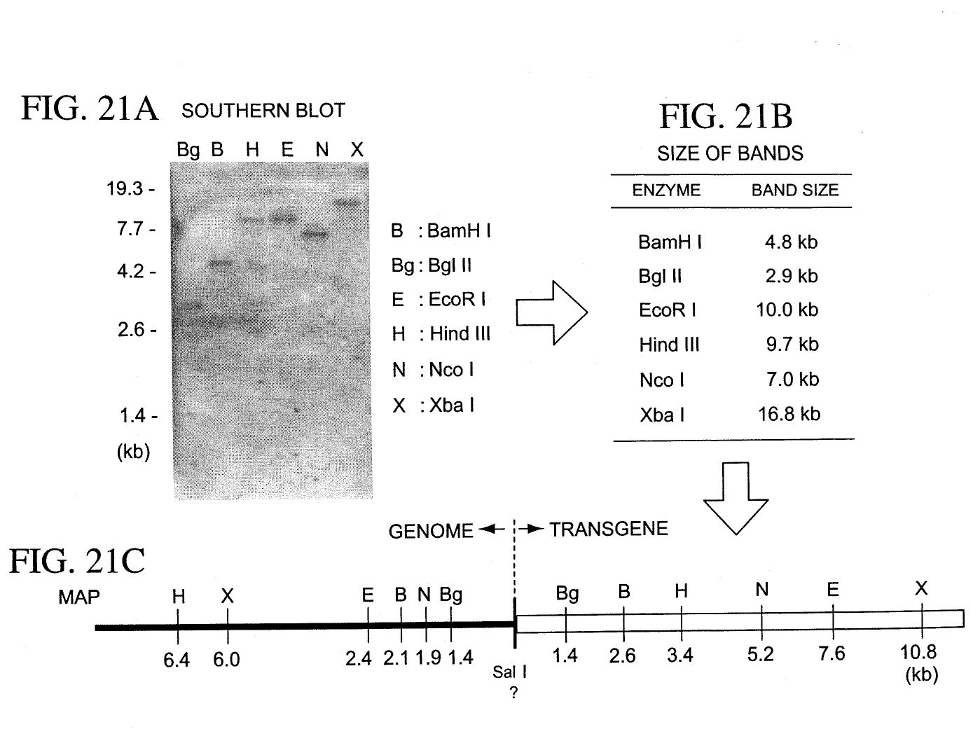

Results of Southern blot analysis are shown in FIG. 21 A. Size of each band was calculated and is shown in FIG. 21 B. The restriction enzyme map was obtained by the information of size of bands and illustrated in FIG. 21C. The map provides the following valuable information. First, asymmetric pattern with respect to the transgene/mouse genome junction point suggests that the transgene does not have a head-to-head configuration. Second, the fact that only a single band was obtained in each restriction enzyme digestion step suggests that a single copy of the transgene should be integrated in the mouse genome in TgPVR21 transgenic mice.

Cloning and Sequencing of the 5′Region of the Transgene/Mouse Genome Junction

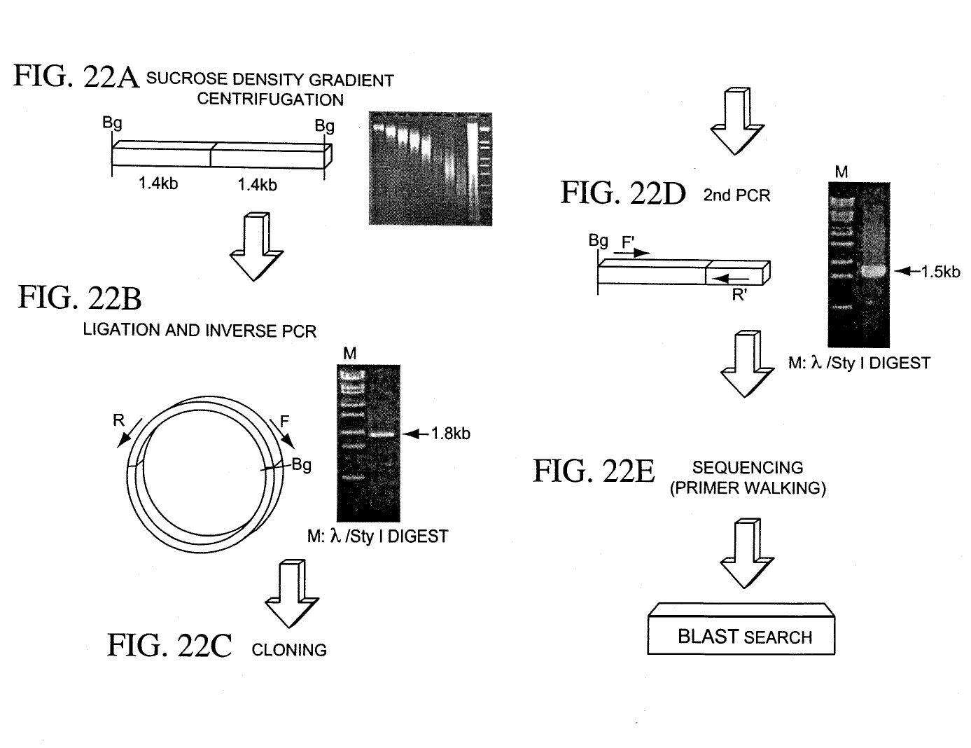

Genomic DNA from a transgenic homozygote of TgPVR21 was completely digested with BglII. DNAs including 2.9 kb fragments were fractionated by ultracentrifugation on sucrose density gradient and subjected to self-ligation (FIG. 22A). Inverse PCR was performed with ligated DNA for amplification of the 5′ genome/transgene junction (FIG. 22B). The PCR products were subjected to direct sequencing to obtain nucleotide sequence information of the junction site (the first PCR). Then, a DNA fragment, including the transgene/mouse genome junction region, was cloned from genomic DNA using the first PCR products as probe (FIG. 22C). The second PCR was performed using the cloned DNA as template, and expected 1.5 kb PCR products were amplified (FIG. 22D). Finally, PCR direct sequencing with walking primers was performed to obtain genome information of a 1 kb upstream from transgene/mouse genome junction point (FIG. 22F).

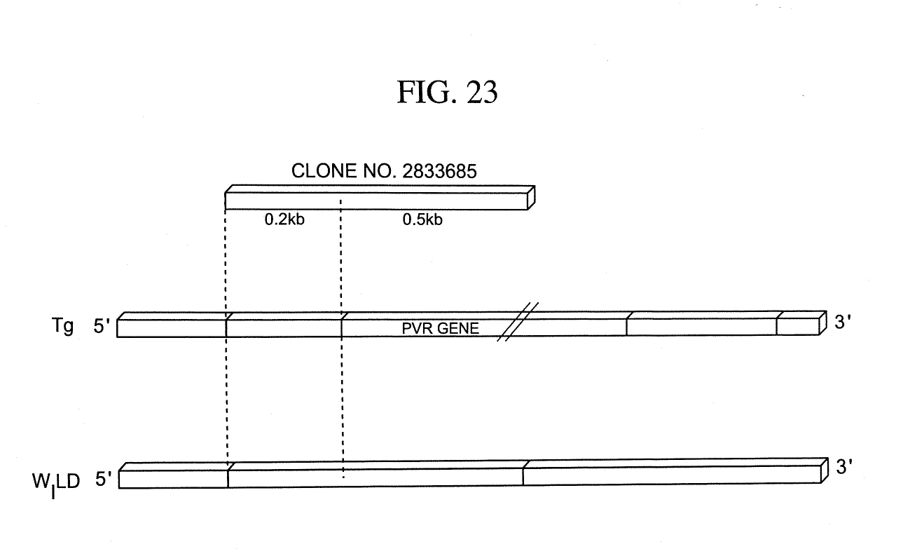

BLAST search was performed with the obtained mouse genome information of the transgenic/mouse genome junction site. The BLAST search revealed a registered clone No. 2833685 having complete homology with 200 bp of the cloned fragment (PVR gene), and the structure of upstream site of the transgene/mouse genome junction region was determined as illustration in (FIG. 23).

### Example 6

### Widening Genetic Background in Order to Achieve Widened Genetic Diversity

If laboratory animals are used in safety tests, it is highly desirable to widen (expand) their genetic background to achieve widened genetic diversity which, in turn, results in a wider range of sensitivity, variety of performance and wider spectrum in phenotypic and dramatypic aspects, Furthermore, reproducibility of the system is not assured without validating the continuous genetic equity and stability of such animals. In order to ensure both widened genetic background and continuous genetic equality/stability, hybrid animals from selected inbred (and completely congenic) strains are produced. When further expansion of genetic diversity if required, hybrid strains can be mate with other hybrid strains in order to produce multi-cross hybrids. In this way, widened genetic diversity is ensured by hybrid-mating, while continuous genetic identity/stability is assured by genetic monitoring of each selected strain. The first step in this process, is the selection of the most suitable background strain for first generation (F1) animals. Since different strains show different sensitivity, spectrum and performance with regard to a target disease, the selection includes review of Information related to the target disease in various strains. Such information is available, for example, from the Jackson Laboratory database (Bar Harbor, Me., U.S.A.), and from experts of the target disease. A second component of the selection of background strains is the review of information available about the reproductive index of various strains. Such information is available from the Reproductive Index Database of Central Institute for Experimental Animals (CIEA) of Japan.

In general, the goals of F1 selection from several inbred strains are the preservation of the diversity of the target disease (e.g. incidences and spectrum of carcinoma), similarly to the diversity observed in human patients, and the provision of stable reproductive ratio, which allows better planning of the number of animals needed.

The reproductive data for various inbred mouse strains are illustrated in the following Table B.

TABLE B

| | Birth- | Average | Weaning | Productive |
|---|---|---|---|---|
| Strain | rate % | of sib | ratio | index |
| C57BL/6J | 84.8 | 6.2 | 92.3 | 4.8 |
| BALB/cByJ | 88.6 | 6.4 | 95.3 | 5.3 |
| AKR/J | 45.3 | 4.9 | 75.2 | 1.7 |
| C3H/HeN | 52.0 | 5.6 | 89.6 | 2.6 |
| DBA/2J | 88.9 | 4.6 | 91.7 | 3.7 |
| C57BL/6J-TgrasH2 | 43.2 | 6.0 | 89.0 | 2.3 |
| C3H/HeJ | 88.4 | 5.7 | 93.5 | 4.7 |
| DBA/2N | 80.5 | 4.5 | 93.4 | 3.9 |

CIEA and Japan CLEA

Birthrate=% number of mother mice over number of mating parent mice. Average of sib= total number of siblings over the number of mother mouse that delivered. Weaning ratio= the number of siblings that weaned over total number of siblings. Productive index= the number of siblings that weaned over the number of mating parent mice.

TABLE C

| | Birth- | Average | Weaning | Productive |
|---|---|---|---|---|
| X | rate % | of sib | ratio | index |
| BALB/cByJXC57BL/6J | 89.6 | 8.2 | 95.2 | 7.0 |
| CB6F1 | | | | |
| BALB/cByJ#C57BL/6J- | 44.5 | 7.5 | 96.4 | 3.2 |
| TgrasH2 | | | | |
| C57BL/6J#DBA/2J | 76.6 | 9.0 | 96.2 | 6.7 |
| BDF1 | | | | |
| C57BL/6JxC3H/eJ B6C3F1 | 95.6 | 8.2 | 95.8 | 7.5 |

The data set forth in Tables B and C are combined in the following Table D.

TABLE D

| | C57BL/ | BALB/ | AKR/ | C3H/ | DBA/ | B6J- | C3H/ | DBA/ |
|---|---|---|---|---|---|---|---|---|
| | 6J | cByJ | J | HeN | 2J | TgrasH2 | HeJ | 2N |
| C57BL/6J | 4.8 | | | 7.5 | 6.7 | 2.3 | | |
| BALB/cByJ | 7.0 | 5.3 | | | | 3.2 | | |
| AKR/J | | | 1.7 | | | | | |
| C3H/HeN | | | | 2.6 | | | | |
| DBA/2J | | | | | 3.7 | | | |
| B6J-TgrasH2 | | | | | | | | |
| C3H/HeJ | | | | | | | 4.7 | |
| DBA/2N | | | | | | | | 3.9 |

### Example 7

### TgPVR21

Because only primates are susceptible to polioviruses, the neurovirulent safety and consistency of oral polio virus vaccine (OPV) had been traditionally assayed in the monkey neurovirulence test: (MNVT). After the development of transgenic (Tg) mice carrying the gene for human poliovirus receptor (PVR), the suitability of these mice to replace monkeys for OPV testing was evaluated. Two lines of Tg mice, TgPVR1 and TgPVR21, were tested. The TgPVR21 mice, inoculated in the spinal cord, were as sensitive as monkeys in discriminating between type-3 and type-2 OPV lots that had passed and those that had failed the monkey neurovirulence test. Results of the new molecular assay by polymerase chain reaction and restriction enzyme cleavage indicated that each OPV lot contained minuscule amounts of neurovirulent revertants in the viral genome. All type-3 OPV lots that failed the monkey neurovirulence test had higher percentages of 472-C revertants than did lots that passed this test. Analysis of multiple type-3 OPV lots also indicated a good correlation between the contents of 472-C revertants and results of the TgPVR21 mouse test. An overview of a significant set of data suggests that the TgPVR21 mouse model is suitable for the evaluation of type-3 and type-2 OPV. The necessity of the TgPVR mouse test for the neurovirulence of type-1 OPV, which is the most stable of the three Sabin strains, is under consideration.

Only primates are susceptible to all three serotypes of poliovirus, so the safety of oral poliovirus vaccine (OPV) and its consistency have been tested in the monkey neurovirulence test (MNVT) (1). About 100 monkeys are used for each trivalent vaccine batch. In a number of countries the MNVT is preformed twice, once by the manufacturer and once by the national control authority. In addition to the high cost, monkeys are usually obtained from the wild with a potential for transmitting exotic diseases to humans. We describe the status of an alternative animal system-transgenic mice susceptible to poliovirus.

Two groups of scientists derived transgenic mice carrying the human poliovirus receptor (TgPVR) mice by introducing into the mouse genome a human gene encoding the cellular receptor to poliovirus (Ren et al., Cell 63: 353-362, 1990; Koike et al. Proc. Natl. Acad. Sci. USA 88: 951-955, 1991). When infected with poliovirus. TgPVR mice developed flaccid paralysis, followed by the death of some mice, and histologic lesions in the central nervous system, similar to those observed in monkeys. The TgPVR mice have been widely used to study various aspects of the pathogenesis of experimentally induced poliomyelitis and poliovirus attenuation (Ren et al. Cell 63: 353-362.1990; Koike et al., Proc. Natl. Acad. Sci. USA 88: 951-955, 1991; Ren et al., J. Virol. 65: 1377-1382, 1991; Ren et al., J. 66″296-304, 1992; Racamello et al. Develop. Biol. Stand. 78: 109-116, 1993; Koike et al., Develop. Biol. Stand 78: 101-107.1993; Horie et al, J. Virol. 68: 681-688, 1994; Koike et al. Arch. Virol. 139: 351-362, 1994). In 1992 the World Health Organization (WHO) recommended a comparison of the sensitivity of TgPVR mice (Koike et al., Proc. Natl. Acad. Sci. USA 88: 951-955, 1991) with that of monkeys by use of type-3 poliovirus strains with different degrees of neurovirulence (World Heath Organization, Bull. W. H. O. 21: 233-237, 1992). A study conducted by the U.S. Food and Drug Administration (FDA) on the TgPVR1 mouse line inoculated intracerebrally indicated that this mouse system could differentiate among the wild-type Leon/37 strain, the Sabin 3 vaccine strain, and a substantially de-attenuated clone of the vaccine virus isolated from stool (Dragunsky et al. Biologicals 21: 233-237, 1993). However, intracerebrally inoculated TgPVR1 mice did not differentiate between OPV lots that passed and those that failed the MNVT. Later Horie et al. (Horie et al, J. Virol. 68: 681-688, 1994) found that this mouse system failed to distinguish between poliovirus type-3 strains with relatively low, but different, levels of neurovirulence for monkeys; OPV lots were not included in their study. Because the TgPVR1 mouse system was unsuitable for testing OPV, attention was given to another mouse line, TgPVR21. Virus samples were inoculated into the mouse spinal cord as in the MNVT. The pass/fail decision on a vaccine batch in the MNVT is based on scoring the histologic lesions in the central nervous system of monkeys (World Health Organization. WHO Tech. Rep. Ser. 800; Appendix 3, annex 1990). By contrast, the test on TgPVR21 mice to detect those OPV lots that failed the MNVT was made possible by the evaluation of clinical signs of poliomyelitis. Encouraging results led to a collaborative study launched by WHO in 1993 (World Health Organization, WHO/MIM/PVD/94.1 World Health Organization. Geneva, 1993). The goal of the study was to evaluate the suitability of TgPVR21 mice for replacing monkeys in the MNVT, first for type-3 OPV. Investigators at the Central Institute for Experimental Animals succeeded in developing TgPVR21 mice from a limited research tool into a reliable supply of animals available in large quantities and with defined quality standards (Hioki et al., Exp. Anim. 42: 300-303 (in Japanese), 1993). Recommendations for the maintenance, containment, and transportation of TgPVR mice were given in the WHO memorandum on transgenic mice susceptible to human viruses (World Heath Organization, Bull. W. H. 0. 71: 497-502, 1993). The inoculation procedure, the clinical scoring method, and the principles of statistical analysis were described (Abe et al., Virology 206: 1075-1083, 1995; Abe et al., Virology 210: 160-166, 1995; Dragunsky et al., Biologicals 24: 77-86, 1996). Virus samples used in those studies were first tested in the MNVT and examined for the abundance of neurovirulent revertants in the viral genome with a very sensitive molecular assay by polymerase chain reaction and restriction enzyme cleavage developed at the FDA (Chumakoc et al., Proc. Natl. Acad. Sci. USA 88: 199-203.1991). The latter method detected minuscule amounts of revertants at position 472 (U→C) and greater amounts at position 2493 (C→U) in each monovalent type-3 OPV lot. The 472-C reversion in type-3 OPV has been documented as a key contributor to increased neurovirulence in the MNVT. Vaccine lots that failed the MNVT contained >1% of these revertants. Back-mutations homologous to those at position 472 in type-3 OPV also were found in type-1 and type-2 OPV lots, but their contributions to neurovirulence were not as strong (Rezapkin et al., Virology 202; 370-378, 1994; Taffs et al. Virology 209: 366-373.1995). There may be other mutations responsible for neurovirulence that occur in the genomes of the Sabin type-1 and type-2 viruses.

To determine whether TgPVR21 mice can detect type-3 vaccine lots that failed the MNVT, the WHO study involved one vaccine lot that contained 3% 472-C revertants in comparison with the reference vaccine WHO/III, which contained 0.5% 472-C revertants. Results from all the participating laboratories indicated that TgPVR21 mice clearly discriminated between the two vaccines (Wood, D. J., Vaccine (in press), 1996). The discrimination was better when clinical scores and the day of the appearance of clinical signs of infection (i.e., failure time) were used as a criteria. Fifty percent paralytic dose and 50% lethal dose were less satisfactory. The majority of the type-3 OPV preparations that failed the MNVT contained <3% 472-C revertants, most of them <2%. For the sake of brevity, the latter vaccines were named “marginal”. Three marginal vaccines (1.3, 1.4, and 1.7%) were tested at the FDA along with the WHO/iii and NC-2 (0.5 and 0.8% 472-C revertants respectively) (Dragunsky et al., Biologicals 24: 77-86, 1996). All three marginal lots failed the mouse test with high probability values for the two main indicators of neurovirulence, clinical scores and failure time. One more vaccine lot that contained only 1.4% 472-C revertants and passed the MNVT failed the mouse test, a finding which might suggest a higher sensitivity of the mouse test than the MNVT.

An interesting question was the relationship between the content of 2493-U revertants in a vaccine or an experimental sample and the neurovirulence in moneys and TgPVR21 mice. Reports on the role of these revertants in neurovirulence for monkeys were controversial (Tatem et al. J. Virol. 66: 3194-3197, 1992; Chumakov et al. J. Virol. 66: 966-970, 1992). The first report considered back-mutation at this position as the most important in increased neurovirulence for monkeys, whereas the findings in the second publication indicated otherwise. Mutations at this position develop faster than those at position 472. Therefore vaccine lots with some increase in the percentages of 472-C usually have very high content of 2493-U, up to 100%. Some manufacturers produced vaccines derived form the Sabine 3 clones which have 100% 2493-U revertants and a very low content (0.3%) of 472-C. No data indicate that these vaccines were less safe for humans than vaccines with a low content of 2493-U revertants. One of them, a reference vaccine F313 compared in the MNVT with WHOSE and the NC-2 reference, was no more virulent than those two vaccines (16, Dragunsky et al., Biologicals 24: 77-86, 1996). However, in TgPVR21 mice, F313 had a higher level of neurovirulence than WHO/III and NC-2 (Dragunsky et al., Biologicals 24: 77-86, 1996). It became essential to determine whether the TgPVR21 mouse test can discriminate between F313 and its derivatives with an increases content of 472-C revertants, which would mimic “bad” vaccines. Therefore two experimental passage samples derived from the F313 vaccine and containing 1.8 and 2.4% 472-C were tested in mice against the parental F313 vaccine. The TgPVR21 mouse test differentiated among these samples (Dragunsky et al. Biologicals 24: 77-86, 1996). Abe et al. (Abe et al. Virology 210: 160-166, 1995) inoculated TgPVR21 mice with WHO/111 and F313 references and compared them with two F313-derived preparations grown at 38° C. They observed close correlations of the MNVT and mouse test results. Unfortunately the two viral preparations grown at 38° C. could not be considered similar to bad vaccines because they contained 78 and 94% 472-C revertants and had changed an in vitro temperature sensitivity marker of attenuation from rct40− to rct40+. This indicated higher neurovirulence than could occur in a vaccine under manufacturing conditions. According to the requirements for OPV production (World Health Organization, WHO Tech. Rep. Ser. 800: 46-49, 1990), vaccine virus growth in cell culture must not exceed 35.5±5° C. Higher temperatures cause selective growth of more neurovirulent viral particles.

During the OPV-3 study in TgPVR21 mice the most discriminating virus doses in all the experiments were 3.5 and 4.5 login of a 50% tissue culture infective dose (TCID₅₀). It was found that reliable discrimination of marginal vaccines could also be achieved by using only these two doses but increasing the number of mice inoculated with each dose. Besides a sufficient number of mice per group, another factor is critical for success; 1.0 logic TCID₅₀ difference in the virus content in the inocula for the MNVT does not matter (Contrearas et al., J. Biol. Stand. 16: 195-205, 1988). By contrast, a stronger dose dependence in the mouse test and the very small volume of the inoculum (0.5 μl) are the most likely reasons for the difference between the mouse and monkey tests. To achieve the necessary precision and to harmonize results between laboratories, it was recommended that the titration assay method described in the WHO guidelines be followed (World Health Organization, Document WHO/BLG/95.1, Chap. 9, p. 67-74, World Health Organization, Geneva, 1995).

In experiments with type-2 OPV conducted at the FDA (Dragunsky et al., Biologicals 24: 77-86, 1996) TgPVR21 mice were inoculated with three vaccine lots that passed and two lots that failed the MNVT, along with the type-2 reference vaccine WHO/II. In addition, three experimental samples were derived from a “good” vaccine lot. One of these samples passed and two failed the MNVT. The results indicated a good correlation between the MNVT and the TgPVR21 mouse test.

Because no type-1 OPV lot repeatedly failed the MNVT, the FDA used one vaccine lot and one experimental passage preparation that failed the MNVT once but passed on repeated testing (Dragunsky et al., Biologicals 24: 77-86, 1996). Inoculation of TgPVR21 mice into the spinal cord with the vaccine lot and the passage sample failed to discriminate between these two preparations and the U.S. reference vaccine. This negative result might be due rather to the peculiarities of type-1 OPV. First of all, the Sabin 1 strain is the most stable of the three serotypes, and probably there is no “bad” type-1 OPV lot to be tested in mice. In some instances the type-1 vaccine lot would fail the MNVT, but when the test was repeated, it would pass (Marsden et al., J. Biol. Stand. 8: 303-309, 1980; Lovenbook. I., Unpublished data). Some experts even question the necessity of the monkey test for type-1 OPV. Abe et al. (16) obtained samples of type-1 OPV by growing the virus at 38° C. These preparations failed the MNVT and TgPVR21 mouse tests, and the rct40 marker was changed from negative to positive, indicating again, as in their work with type 3 (Abe et al., Virology 210: 160-166.1995), that the neurovirulence of the samples was higher than would have been expected for any bad vaccine. The fact that for these preparations there was a correlation between the MNVT and the TgPVR21 mouse test strengthens the point that failure with the TgPVR21 mouse test for type-1 OPV might be due not to the unsuitability of the mouse model but to the stability of the Sabin 1 strain when it is grown under manufacturing conditions.

An overview of a substantial body of data that has accumulated during the past several years suggests that spinal core-inoculated TgPVR21 mice provide a suitable model for evaluation of the neurovirulence of type-3 and type-2 OPV. This mouse model can be considered as a possible replacement for monkeys. The applicability of the mouse test for type-1 OPV has yet to be resolved. The established production of TgPVR mice, their pathogen-free health status, and lower cost relative to monkeys make them highly appealing for the neurovirulence testing of OPV.

### Example 8

### Tg-cHa-ras

Rapid carcinogenicity tests were done with transgenic (Tg) mice human prototype c-HRAS gene, namely BALB/cByJ x C57BL/6JFl-TgN (HRAS) 2 or CB6F1-F1RAS2 mice. The studies were conducted as the first step in the evaluation of the CB6F1-HRAS2 mouse as a model for the rapid carcinogenicity testing system. Results of the short-term tests of various genotoxic carcinogens indicated that CB6F1-HRAS2 mice are more susceptible to these carcinogens than control non-Tg mice. According to the first-step evaluation studies, more rapid onset and higher incidence of more malignant tumors can be expected with a higher probability after treatment with various genotoxic carcinogens in the CB6F1-HRAS2 mice than in control non-Tg mice. The CB6F1-HRAS2 mouse seems to be a promising candidate as an animal model for the development of a rapid carcinogenicity testing system.

Although continuous effort has been made to conquer cancer not only though approaches from basic and clinical medicine but also through approaches from public health, cancer still remains as the top-ranking cause of death in many countries. Many human cancers are believed to be caused by exposure to environmental chemical carcinogens. To reduce the risk, extensive efforts have been made to identify and eliminate carcinogens. Epidemiologic studies and carcinogenicity tests with experimental animals are used to identify human carcinogens. Although epidemiologic studies are very reliable and are probably the only way to confirm human carcinogens, this approach is so retrospective that identification of carcinogens can be made only after many victims have appeared.

Carcinogenicity tests are indispensable when one is evaluating the safety of drugs in the process of development and when one is identifying environmental carcinogens. Current carcinogenicity tests with experimental animals do not always have relevance for human risk assessment; mice and rats are generally used because of their short life span and small size. Since a rodent carcinogenicity test extends for >2 years and requires a large number of animals, it demands a large space for animal testing, a large number of laboratory technicians, and enormous cost. When positive results are obtained in the carcinogenicity tests, it is not unusual for one to realize that time, effort, and cost for the development of the new drug have been wasted. Moreover, there are many chemicals in our environment that have not been tested, and thousands of new chemicals are synthesized every year. There is a clear need to improve the process of carcinogen identification so that more chemicals can be evaluated. Therefore the development of rapid carcinogenicity testing systems that can evaluate carcinogenicity within a short period is essential to improve efficiency in the development of new drugs and the identification of environmental carcinogens.

To develop rapid carcinogenicity testing systems, animals that are susceptible to carcinogens are indispensable. Transgenic (Tg) animals harboring a proto-oncogene and/or animals lacking a tumor-suppressor gene are expected to be more susceptible to various carcinogens than normal animals, since carcinogenesis is a multi-stage process driven by genetic and epigenetic damage in susceptible cells that gain a selective growth advantage and undergo clonal expansion, probably as the result of activation of proto-oncogenes and/or inactivation of tumor-suppressor genes.

The ras family genes are involved in the regulation of cell proliferation and are activated by somatic point mutations in various human tumors (Lowy et al., Annu. Rev. Biochem. 62: 851-891, 1993; Bos, J. L., Cancer Res. 49; 4682-4689, 1989; Anderson et al. Environ. Health Perspect 98: 13-24, 1992) as well as in experimental animal models (Anderson et al., Environ. Health Perspect 98: 13-24, 1992; Guerrero et al., Mutat. Res. 185: 293-308, 1987). Activation of the ras family genes by point mutations is observed in approximately 30% of human tumors. Therefore, the Tg mouse carrying the human c-HRAS gene may be a candidate as an animal model for rapid carcinogenicity testing.

Collaborative evaluation studies on the usefulness and limitations of Tg mice carrying the human c-HRAS gene as an animal model for rapid carcinogenicity testing are now under way at our institutions, at several Japanese pharmaceutical companies and at the U.S. National Institute of Environmental Health Sciences (NIEHS) (Drs. R. R. Maronpot and R. W. Tennant). To evaluate the usefulness and limitations of Tg mice, a system for the mass production and supply of genetically and microbiologically defined Tg mice is indispensable. In this overview we introduce our current evaluation studies carried out by investigating the carcinogenic response of Tg mice carrying the c-HRAS gene to various carcinogens and compare the response with that of control nontransgenic (non-Tg) mice and the results of 2-year bioassay.

Characteristics of Tg mice carrying the human prototype c-HRAS gene: The Tg mice carrying the prototype human c-HRAS gene were originally established by Katsuki and his colleagues at the Central Institute for Experimental Animals (CIEA) (Saitoh et al., Oncogene 5: 1195-1200, 1990); the mice carry this gene with its own promoter region, which encodes the prototype c-HRAS gene product (i.e., p21) with no capacity of transforming NIH3T3 cells (Saitoh et al., Oncogene 5: 1195-1200, 1990). Five or six copies of human c-HRAS gene are integrated into the genome of each Tg mouse in a tandem array (Saitoh et al., Oncogene 5: 1195-1200, 1990). Transgenes are expressed in the tumors and in normal tissues, and the total amount of p21 detected by immunoblot analysis is two to three times higher in Tg mice than in non-Tg mice (Saitoh et al., Oncogene 5: 1195-1200, 1990). No mutations of the transgenes are detected in the normal tissues of the Tg mice (Saitoh et al., Oncogene 5: 1195-1200, 1990). Approximately 50% of the rasH2 mice (C57BL/6 X BALB/cF2) develop spontaneous tumors within 18 months after birth (Saitoh et al. Oncogene 5: 1195-1200, 1990). About 60% of the tumor-bearing mice have angiosarcomas (Saitoh et al. Oncogene 5: 1195-1200, 1990). Lung adenocarcinomas, skin papillomas, Harderian gland adenocarcinomas, and lymphomas are also seen at 18 months of age, but with much lower incidence (Saitoh et al., Oncogene 5: 1195-1200, 1990). However, neither tumors nor preneoplastic lesions are observed in F2 transgenic offspring of rasH2 mice at 6 months of age (Saitoh et al., Oncogene 5: 1195-1200, 1990).

The genetic background of CB6F1-HRAS2 mice used in this study was F1 of transgenic male C57BL/6J and female BALB/cByJ mice. Transgenic male C57BL/6J mice were established by backcrossing rasH2 mice more than eight times with C57BL/6J mice. The C57BL/6J males carrying the transgene were crossed with BALB/cByJ female mice. The F1 offspring were screened by polymerase chain reaction or Southern blot analysis for the presence of the human prototype c-HRAS gene. The FT mice carrying the human c-HRAS gene, namely BALB/cByJ x C57BL/6JF1-TgN(HRAS) 2 (CB6F1-HRAS2) mice produced at the CIEA, 7 to 9 weeks of age, were used for carcinogenicity tests. Among the littermates, mice (CB6F1) not carrying the human c-HRAS gene were used as non-Tg controls. Since a large number of CB6F1-HRAS2 mice are required in the form of standardized laboratory animals in this study, practical development is necessary. The concept and system used in this development are described in detail by Nomura in the first overview of this issue.

The body weight of male and female CB6F1-HRAS2 mice was 80 to 90%> of that corresponding non-Tg mice. As for the organs tested (brain, thyroid gland, heart, lung, liver, spleen, kidney, adrenal glands, testes, and ovaries), the organ to body weight ratios of the Tg mice were similar to those of non-Tg mice. Blood biochemical and hematologic data were not significantly different between Tg and non-Tg mice. The survival rate of male and female CB6F1-HRAS2 mice at 77 weeks of age was 53% and 32% respectively. Approximately 50% of the CB6F1-HRAS2 mice died of angiosarcoma, and approximately 20% of the dead animals bore lung adenocarcinomas and/or lung adenomas, consistent with the previous results in rasH2 mice (Saitoh et al., Oncogene 5: 1195-1200, 1990). In this study only a few spontaneous lung adenomas but no other spontaneous tumors were observed in the CB6F1-HRAS2 mice during the 6-month carcinogenicity experiments, which were terminated at the latest by 35 weeks of age (survival rate of CB6F1-HRAS2 mice at 35 weeks of age was 295%). The low incidence of spontaneous tumors in CB6F1-HRAS2 mice allows us to use this mouse as a tool for rapid carcinogenicity testing.

Rapid carcinogenicity tests: These studies on rapid carcinogenicity testing have been done at our institutions and at several Japanese pharmaceutical companies (Table 1).

TABLE 1
Results of rapid carcinogenicity tests with CB6F1-HRAS2 mice in Japan

| | | Route of | Genotoxicity | Rapid tumor | Tumor incidence | Malignant Tumors | |
|---|---|---|---|---|---|---|---|
| Tested Chemicals | Dose | administration | (Salmonella) | response in Tg mice | and/or multiplicity | Tg | Non-Tg |
| 4NQO^{1,a} | 15 mg/kg × 1 | s.c. | + | + | Tg > non-Tg | + | − |
| MNNG^{2,a} | 2.5 mg × 1 | Gavage | + | + | Tg > non-Tg | + | − |
| MNU^{3,a} | 75 mg/kg × 1 or | i.p. | + | + | Tg > non-Tg | + | − |
| | 15 mg/kg × 5 | | | | | | |
| Vinyl carbamate^{1,6,b} | 60 mg/kg × 1 | i.p. | + | + | Tg > non-Tg | ++ | + |
| Den^{4,A} | 90 mg/kg × 1 | i.p. | + | + | Tg > non-Tg | + | − |
| MAM^{5,a} | 20 mg/kg × 1/w for 6 wk | s.c. | + | +^{c} | Tg > non-Tg^{c} | + | − |
| Cyclophosphamide^{1,a} | 30 mg/kg × 2/w for 25 wk | Gavage | + | +^{c} | Tg ÷ non-Tg | + | − |
| 4HAQO^{5,b} | 10 or 20 mg/kg × 1 | i.v. | + | + | Tg > non-Tg | ++ | + |
| Ethylene thiourea^{1,b} | 0.3% | Feed | − | +^{c} | Tg ÷ non-Tg | + | + |

4NQO = 4-Nitroquinoline-1-oxide; MNNG = N-Methyl-N′-nitro-N-nitrosoguanidine; MNU = N-Methyl-N′-nitrosourea; DEN = N-Methyl-N′-nitrosourea; MAM = Methylazoxymethanol; 4HAQO = 4-Hydroxyaminoquinoline-1-oxide.
¹National Institute of Health Sciences (NIHS).
²Yamanouchi Pharmaceutical Co., Ltd.
³Chugai Pharamceutical Co., Ltd.
⁴Sankyo Co., Ltd.
⁵CIEA.
⁶U.S.-Japan collaborative study.
^{a}Reference 9;
^{b}unpbulished data;
^{c}statistically not significant

4-Nitroquinoline-1-oxide (4NQO), a water-soluble genotoxic carcinogen, is known to induce squamous cell carcinomas of the skin (Nakahara et al., Gann 48: 129-136, 1957) and oral cavity (Hawkins et al. Head Neck 16: 424-432, 1994), and lung tumors (Inayama, Y., Jpn. J. Cancer Res. 77: 345-350, 1986) in mice. Approximately 90% of 4NQQ-treated CB6F1-HRAS2 mice (male and female) bore skin papillomas 16 weeks after a single subcutaneous (s. c.) injection of 15 mg of 4NQO/kg of body weight (Yamamoto et al., Carcinogenesis 17: 2455-2461, 1996). Squamous cell carcinomas of skin were observed only in 4NQO-treated CB6F1-HRAS2 mice, not in control non-Tg mice. No skin tumors were observed in 4NQO-treated non-Tg mice and in vehicle-treated animals. The 4NQO also induced lung tumors. Lung adenocarcinomas were observed only in 4NQO-treated CB6F1-HRAS2 mice, not in corresponding non-Tg mice (Yamamoto et al., Carcinogenesis 17: 2455-2461.1996). The incidence of lung adenoma in 4NQO-treated CB6F1-HRAS2 mice also was higher than that in corresponding non-Tg mice (Yamamoto et al. Carcinogenesis 17: 2455-2461, 1996).

Cyclophosphamide, an anti-neoplastic agent, is carcinogenic in rodents and humans (International Agency for Research on Cancer, IARC vol 26, p 165-202, Lyon, France, 1981). The major target organs are the bladder, lung, mammary gland, and lymphatic systems (International Agency for Research on Cancer, IARC vol 26, p 165-202, Lyon, France, 1981). Chronic oral administration of either 10 or 30 mg of cyclophosphamide/kg twice a week for 25 weeks induced lung tumors in CB6F1-HRAS2 and non-Tg mice (Yamamoto et al., Carcinogenesis 17: 2455-2461, 1996). Adenocarcinomas were observed only in one cyclophosphamide-treated male CB6F1-HRAS2 mouse but not in corresponding non-Tg mice or in vehicle-treated animals. The incidence of lung adenoma in cyclophosphamide-treated CB6F1-HRAS2 mice was not significantly different from that in corresponding non-Tg mice. No tumor was observed in other organs such as the bladder, mammary gland, and lymphatic systems (Yamamoto et al., Carcinogenesis 17: 2455-2461, 1996).

N-methyl-N′-nitro-N-nitrosoguanidine (MNNG) is an alkylating agent and is carcinogenic in various species of animals including the mouse (International Agency for Research on Cancer. IARC vol 4, p 183-195. Lyon, France, 1974). The forestomach and esophagus are target organs of MNNG after its oral administration (International Agency for Research on Cancer, IARC vol 4, p 183-195, Lyon, France, 1974). A single oral administration of 2.5 mg of MNNG/mouse induced forestomach papillomas in 100% of male and female CB6F1-HRAS2 mice, whereas only 11% of female and 0% of male non-Tg mice developed papillomas 13 weeks after MNNG treatment (Yamamoto et al., Carcinogenesis 17; 2455-2461, 1996). Even at 26 weeks after MNNG administration, squamous cell carcinomas were observed only in MNNG-treated CB6F1-HRAS2 mice but not in corresponding non-Tg mice (Yamamoto et al., Carcinogenesis 17: 2455-2461, 1996).

N-Methyl-N-nitrosourea (MNU) is carcinogenic in various species of animals and induces tumors at various sites such as skin, forestomach, lymphatic system, and lung (International Agency for Research on Cancer, IARC vol 17, p 117-255, Lyon, France, 1978). Intraperitoneal (i. p.) injection of MNU, either once at the dosage of 75 mg/kg or five times (once a day for 5 consecutive days) at the dosage of 15 mg/kg, induced various types of tumors in CB6F1-HRAS2 mice (Yamamoto et al., Carcinogenesis 17: 2455-2461.1996). A significantly high incidence of skin papilloma was seen in CB6F1-HRAS2 mice after MNU treatment, compared with that in corresponding non-Tg mice (Yamamoto et al., Carcinogenesis 17: 2455-2461, 1996). The MNU induced skin papillomas in CB6F1-HRAS2 mice at a high incidence but did not induce skin papillomas and hyperplasias in non-Tg mice, at least during the 14 weeks of observation. The MNU-treated CB6F1-HRAS2 mice also developed forestomach papillomas at a high incidence, whereas MNU-treated non-Tg mice developed no papillomas (Yamamoto et al., Carcinogenesis 17: 2455-2461.1996). Forestomach squamous cell carcinoma also was seen only in MNU-treated CB6F1-HRAS2 mice but not in non-Tg mice. An do et al. (Ando, et al., Cancer Res. 52: 978-982, 1992) reported a higher incidence of forestomach and skin papillomas in rasH2 mice after single i. p. injection of MNU, compared with corresponding non-Tg mice. The incidence of lymphoma was higher in male CB6F1-HRAS2 mice treated once with 75 mg of MNU/kg, compared with the response in the corresponding non-Tg mice (Yamamoto et al., Carcinogenesis 17: 2455-2461, 1996).

N,N-Diethylnitrosamine (DEN) is carcinogenic in various animal species (International Agency for Research on Cancer, IARC vol 17, p 83-124, Lyon, France, 1978). The major target organs of DEN are the liver, lung, and forestomach (International Agency for Research on Cancer. IARC vol 17, p 83-124, Lyon. France, 1978). A single i.p. injection of 90 mg of DEN/kg caused forestomach squamous cell carcinomas and lung adenocarcinomas only in CB6F1-HRAS2 mice as early as 3 months after DEN administration (Yamamoto et al., Carcinogenesis 17: 2455-2461, 1996). Six months after DEN administration the incidence of both types of malignant tumors in CB6F1-HRAS2 mice increased substantially (Yamamoto et al., Carcinogenesis 17: 2455-2461, 1996). These tumors were never observed in DEN-treated non-Tg mice during the 6-month observation period. The incidence of lung adenoma in CB6F1-HRAS2 mice was similar to that in non-Tg mice at 3 months after DEN administration. Six months after DEN administration the incidence of adenoma was significantly higher in non-Tg mice than in CB6F1-HRAS2 mice, corresponding to the increased incidence of lung adenocarcinoma in the CB6F1-HRAS2 mice (Yamamoto et al., Carcinogenesis 17: 2455-2461, 1996).

Vinyl carbamate, a metabolite of urethane, is known to induce lung and liver neoplasms (Massey et al., Carcinogenesis 16: 1065-1069, 1996; Maronpot et al., Toxicology 101: 125-156, 1995). A single i. p. injection of 60 mg of vinyl carbamate/kg induced lung adenomas and adenocarcinomas in 100% and 50%) of CB6F1-HRAS2 mice respectively, 16 weeks after the carcinogen administration (Maronpot et al. manuscript in preparation). Although non-Tg mice also developed lung adenomas at >90% incidence, tumor multiplication was lower than that in the corresponding CB6F1-HRAS2 mice. The incidence of lung adenocarcinoma was much lower in non-Tg mice than in CB6F1-HRAS2 mice. Approximately 90% of the latter bore spleen hemangiosarcomas, but none developed in non-Tg mice.

Methylazoxymethanol (MAM) is carcinogenic in rodents and induces colon tumors (Reddy et al., J. Natl. Cancer Inst. 71: 1181-1187, 1984; I? eschner et al., J. Cancer Res. Clin. Oncol. 115: 335-339, 1989), lung tumors (Reddy et al., J. Natl. Cancer Inst. 71: 1181-1187, 1984), and perianal squamous cell carcinomas (Kumagai et al., Gann 73: 358-364, 1982). One s. c. injection of 20 mg of MAM/kg a week for 6 weeks caused skin papillomas, colon adenomatous polyps, squamous cell carcinomas of the rectum, and stomach papillomas in CB6F1-HRAS2 mice but not in non-Tg mice 24 weeks after the initial MAM administration (Yamamoto et al., Carcinogenesis 17: 2455-2461, 1996). Skin papillomas were restricted to the anus and scrotum, consistent with the previous report in non-Tg mice of a different strain (Kumagai et al., Gann 73: 358-364, 1982). A similar lung adenoma incidence was observed in CB6F1-HRAS2 and non-Tg mice treated with MAM.

A single intravenous (i. v.) administration of 4-hydroxy-aminoquinoline-1-oxide (4HAQO, 10 or 20 mg/kg), a genotoxic carcinogen, induced forestomach and skin papillomas in CB6F1-HRAS2 mice, but these tumors were hardly observed in non-Tg mice, at least within 26 weeks after carcinogen treatment. Although the incidence was low, other tumors (e.g., leukemias and thymomas) were observed only in the Tg mice. Neither the 4HAQO-treated Tg nor the non-Tg mice developed tumors in the exocrine portion of the pancreas, which has been suggested to be a target tissue of this carcinogen (Rao et al., Int. J. Pancreatol. 2: 1-10, 1987). The results of these rapid carcinogenicity tests are summarized in Table 1 above, and the list of chemicals used for rapid carcinogenicity tests is shown in Table 2.

TABLE 2
List of chemicals for rapid carcinogenicity tests

Salmonella mutagenesis assay-positive carcinogens (trans-species)
4-Nitroquinoline-1-oxide (4NQO)^{a}
Cyclophosphamide^{a}
N-Methyl-N′-nitro-N-nitrosoguanidine (MNNG)^{a}
N-Methyl-N′-nitrosourea (MNU)^{a}
N,N-Diethylnitrosamine (DEN)^{a}
Methylazoxymethanol (MAM)^{a}
Vinyl carbamate^{b}
4-Hydroxyaminoquinoline-1-oxide (4HAQO)^{c}
Procarbazine^{c}
Thiotepa^{c}
3-(N-Methyl-N-nitrosamino)-1-(3-pyridyl)-1-butanone (NNK)^{c}
Phenacetein^{c}
4,4′-Thiodianiline^{c}
4-Vinyl-1-cyclohexene diepoxide^{c}
p-Cresidine^{b}
Cupferron^{c}
Melphalan^{b}
Salmonella mutagenesis assay-negative carcinogens (trans-species)
Ethylene thiourea
1,4-Dioxane^{c}
Ethyl acrylate^{c}
Cyclosporin^{b,c}
Furfural^{c}
Benzene^{c}
Diethylstilbestrol^{c}
Salmonella mutagenesis assay-positive noncarcinogens
p-Anisidine^{b}
8-Hydroxyquinoline^{c}
4-Nitro-o-phenylenediamine^{c}
2-Chloromethylpyridine hydrochloride (2-Picolyl chloride hydrochloride)^{c}
Salmonella mutagenesis assay-negative noncarcinogens
Resorcinol^{b}
Rotenone (mouse)^{c}
Xylenes (mixed)^{c}
Tetraethylthiuram disulfide^{c}
Chemicals in bold type = rapid carcinogenicity tests completed or now under way
^{a}Reference 9;
^{b}U.S.-Japan collaborative study;
^{c}rapid carcinogenicity tests conducted or to be conducted at the CIEA

A Salmonella mutagenesis assay-negative carcinogen, ethylene thiourea, is known to induce thyroid neoplasms in rats and mice National Toxicology program of the National Institute of Environmental Health Sciences. Environ. Health Perspect. 101: 264-266, 1993). Only female mice were used for carcinogenicity tests. Mice were fed diets containing 0.1 or 0.3% of ethylene thiourea for 28 weeks. Ethylene thiourea at a concentration of 0.1% did not induce thyroid tumors in CB6F1-HRAS2 mice or in non-Tg mice, whereas 0.3% ethylene thiourea induced thyroid adenomas in 26 and 20% of the Tg and non-Tg mice respectively. The incidence of thyroid adenocarcinoma was also similar (9% in Tg and 4% in non-Tg mice), and no significant difference was observed between the Tg and non-Tg mice.

Both DEN (Ando, et al., Cancer Res. 52: 978-982, 1992) and vinyl carbamate (Maronpot et al., Toxicology 101: 125-156, 1995) are known as potent inducers of liver tumors. However, neither CB6F1-HRAS2 mice nor control non-Tg mice treated with these compounds developed liver tumors. It has been reported that multiple genetic loci control liver tumor development in mice (Gariboldi et al., Cancer Res. 53: 209-211, 1993; Manenti et al., Genomics 23: 118-124, 1994). The C57BL/6 mice have a relatively low susceptibility to chemically induced hepatocarcinogenesis (Diwan et al., Carcinogenesis 7: 215-220, 1986; Stanley et al. Carcinogenesis 13: 2427-2433, 1992) compared with C3H mice, a strain very susceptible to hepatocarcinogenesis (Diwan et <BR><BR><BR> al., Carcinogenesis 7: 215-220.1986; Dragarn et al. Cancer Res. 51: 6299-6303, 1991). It is known that BALB/e mice are very resistant to hepatocarcinogenesis, and the FT hybrid of female C57BL/6 and male BALB/c mice has a low sensitivity to hepatocarcinogenesis (Maronpot et al., Toxicology 101: 125-156, 1995, Stanley et al. Carcinogenesis 13: 2427-2433.1992). Therefore it seems highly possible that CB6F1 mice, the F1 hybrid of male C57BL/6 and female BALB/c mice, have a relatively low susceptibility to hepatocarcinogenesis.

Activation of the HRAS gene has been detected frequently in liver tumors of some mouse strains such as C3H and B6C3F1 (Maronpot et al., Toxicology 101: 125-156, 1995). However, the frequency of HRAS mutation is very low in liver tumors of B6CF1 mice induced by either DEN or vinyl carbamate (Maronpot et al. Toxicology 101: 125-156, 1995). The mutation of HRAS may contribute significantly to liver tumor induction in mouse strains with a high sensitivity to hepatocarcinogenesis but not in strains with a low sensitivity (Maronpot et al., Toxicology 101: 125-156, 1995).

Rapid tumor responses of skin papillomas/squamous cell carcinomas, forestomach papillomas/squamous cell carcinomas, and some other types of tumors were clearly observed in CB6F1-HRAS2 mice, whereas, irrespective of carcinogen types, the incidence and multiplicity of lung adenoma induced by cyclophosphamide, MNU, DEN, or MAM in CB6F1-HRAS2 mice were not significantly higher than those associated with tumors induced by the corresponding carcinogens in non-Tg mice. There are significant differences in pulmonary tumor incidence among various mouse strains after carcinogen exposure (Malkinson, A. M., Toxicology 54: 241-271, 1989). The results of genetic studies of recombinant inbred lines between A/J (very susceptible to lung carcinogenesis) and C57BL/6J (resistant to lung carcinogenesis) suggested that three genetic loci contribute to the difference in susceptibility to pulmonary tumorigenesis in these strains (Malkinson et al, J. Natl. Cancer Inst. 75: 971-974, 1985). The Ki-ras oncogene has been proposed as one of these susceptibility loci (You et al, Proc. Natl. Acad. Sci. USA. 89: 5804-5808, 1992; Chen et al. Proc. Natl. Acad. Sci. USA 91:1589-1593, 1994), and the pulmonary adenoma susceptibility of each mouse strain (e.g., A/J is susceptible, BALB/c is intermediate, and C57BL/6 is resistant) correlates well with the polymorphism in the Ki-ras gene (Chen et al., Proc. Natl. Acad. Sci. USA 91: 1589-1593, 1994). The CB6F1 mice used in this study may have relatively high pulmonary adenoma susceptibility. On the other hand, lung adenocarcinomas developed only in CB6F1-HRAS2 mice, but none or only few developed in non-Tg mice in response to various carcinogens, indicating that CB6F1-HRAS2 mice have some additional capability to accelerate the malignant progression of lung adenomas compared with control CB6F1 mice.

These results indicate that a more rapid onset and a higher incidence of more malignant tumors can be expected with a higher probability after treatment with various genotoxic carcinogens in the CB6F1-HRAS2 mice than in control non-Tg mice. These initial evaluation studies indicated that the CB6F1-HRAS2 mouse seems to be a promising candidate as an animal model for the development of a rapid carcinogenicity testing system.

Perspectives: Although mutagenicity is a major mechanistic determinant of carcinogenicity, this is neither sufficient nor necessary for carcinogenicity. Approximately one-third of the nonmutagenic chemicals have been shown to be carcinogenic, and approximately one-third of the mutagenic chemicals were not carcinogenic in the 2-year rodent bioassay (Ashby et al., Mutat. Res. 257: 229-306, 1992; Zeiger et al, Environ. Mol. Mutagen. 16 (Suppl. 18): 1-14,1990). It has been proposed that chemicals which induce tumors in two rodent species are less influenced by the genetic variability among different species than the chemicals that induce tumors in only one species (Tennant, R. W., Mutat. Res. 286: 111-118, 1993). Thus trans-species carcinogens seem to be more hazardous for humans than are single-species carcinogens.

As for trans-species carcinogens, we have either completed or are already started rapid carcinogenicity tests of 15 Salmonella mutagenesis assay-positive carcinogens (4NQO, cyclophosphamide, MNNG, MNU, DEN, vinyl carbamate, MAM, 4HAQO, procarbazine, thiotepa, NNK, phenacetin, 4,4′-thiodianiline, 4-vinyl-1-cyclohexene diepoxide, and p-cresidine) and six Salmonella mutagenesis assay-negative carcinogens (ethylene thiourea, 1,4-dioxane, ethyl acrylate, cyclosporin, furfural, and benzene (Table 2). Among these carcinogens cyclophosphamide, procarbazine, thiotepa, phenacetin, cyclosporin, and benzene are classified as human carcinogens (group 1) or are probably carcinogenic in humans (group 2A). We are planning to conduct tests with at least two more salmonella-positive trans-species carcinogens (cupferron and melphalan) and one more salmonella-negative carcinogen (diethylstilbesterol) (Table 2). Melphalan and diethylstilbesterol are classified as human carcinogens. The 6-month carcinogenicity tests of these carcinogens may further evaluate whether this CB6F1-HRAS2 mouse is useful as an animal model for rapid and accurate identification of genotoxic and/or nongenotoxic carcinogens.

Since false-positive errors in human carcinogen identification may hinder appropriate drug development and may cause social embarrassment, overprediction of carcinogenicity should be avoided as much as possible. Therefore it must be clarified whether the CB6F1-HRAS2 mice respond negatively to noncarcinogens. Rapid carcinogenicity tests of one Salmonella mutagenesis assay-positive noncarcinogen (p-anisidine) and one Salmonella mutagenesis assay-negative noncarcinogen (resoreinol) are now under way (Table 2). Hereafter we should concentrate more on Salmonella-positive and Salmonella-negative noncarcinogens. We are planning to conduct studies with at least four Salmonella-positive noncarcinogens (8-hydroxyquinoline, 4-nitro-o-phenylenediamine, and 2-chloromethylpyridine) and three Salmonella-negative noncarcinogens (rotenone, xylenes, tetraethylthiuram disulfide) at the CIEA (Table 2). Six chemicals among the aforementioned have been or will be tested in Japan and the United States simultaneously (Table 3).

TABLE 3
U.S.-Japan collaborative studies on a short-term (26
weeks) carcinogenicity tests with CB6F1-HRAS2 mice

| | | Institute | | |
|---|---|---|---|---|
| Chemicals | Dose and Route of Administration | Japan | U.S. | Status |
| Vinyl carbamate | 60 mg/kg × 1, i.p. | NIHS | NIEHS | Completed |
| p-CREsidine | 0.25%, 0.5%, feed | NIHS | NIEHS | Completed |
| Cyclosporin | 5 mg/kg, 10 mg/kg, 25 mg/kg, × 5/W for 26 W gavage | CIEA | NIEHS | Under Way |
| Resorcinol | 225 mg/kg, × 5/W for 24 W gavage | Industry 1 | NIEHS | Under Way |
| Melphalan | 0.3 mg/kg, 1.5 mg/kg × 1/W for 25 W, i.p. | Industry 2 | NIEHS | To be done |
| p-Anisidine | 0.225%, 0.45%, feed | NIHS | NIEHS | Under Way |

Industry 1 = Yamanouchi Pharmaceutical Co., Ltd.
Industry 2 = Kyowa Hakko Kogyo Co.

The current regulatory requirements for assessment of the carcinogenic potential of chemicals in the European Union, United States, and Japan stipulate long-term rodent carcinogenicity studies in two rodent species. Because of the cost of long-term bioassays, their extensive use of animals, the poor mechanistic basis, and relatively low relevance for human risk assessment, it has been considered in the International Conference on Harmonization of Technical Requirements for the Registration of Pharmaceuticals for Human Use (ICH) whether the need for 2-year, carcinogenicity tests with two rodent species could be reduced without compromising human safety. Recent studies on the validation for use of either p53-knockout mice or TG. AC mice (v-Ha-ras transgenic mice) as short-term bioassay models for carcinogen identification have been conducted by Tennant and his colleagues at the NIEHS (Tennant et al., Environ. Health Perspect. 103: 942-950, 1995). At present among various transgenic animals, p53-knockout mice, CB6F1-HRAS2 mice, and TG. AC mice seem to be the most promising candidates for the short-term bioassay models for identifying chemical carcinogens, since a considerable amount of data which indicate possible usefulness have already been accumulated. Although the usefulness and limitations of rapid carcinogenicity testing systems using Tg mice have not been fully evaluated yet, the use of Tg mice to detect potential carcinogens is a topic of discussion as part of the guidelines for ICH. Present 2-year carcinogenicity tests with two rodent species will be replaced by 2-year tests with one species, probably rats, plus short-term bioassays and mechanistic studies.

### Example 9

### Reproducible and Repeatable Carcinogenic Response in rasH2 Mice

To confirm that the rasH2 mouse model possesses reproducible and repeatable performance at dramatype level, carcinogenic response to certain carcinogens was examined in multiple institutions, and incidences of tumor development were compared among institutions.

### Materials and Methods

Carcinogen: N-methyl-N-nitrosourea (MNU), an alkylating agent and genotoxic carcinogen, was used as a positive control carcinogen. Mice in the positive control group were given a single i. p. injection of 75 mg/kg of MNU dissolved in citrate-buffered saline (pH 4.5). The dose of 75 mg/kg was established based on a previous dose finding study.

Mouse: In 1997, mice in the nuclear colony of rasH2 strain were backcrossed to C57BL/6 and generation of backcrossing was beyond N14. In this study, CB6F1-Tg-rasH2 mice produced during 1997 to 1999 at the Central Institute for Experimental Animals (CIEA) were used.

Institutions: Mice were supplied to 11 different institutions (Sankyo, Tanabe, Eisai, Teikoku-Zoki, Daiichi, Shionogi, Dainippon, Mitsubishi, Fujisawa, Wyeth, and Shinyaku). All work at each institution was conducted by Usui of CIEA as the ILSI. ACT (International Life Sciences institute, Alternative to Carcinogenicity Testing) project.

### Results

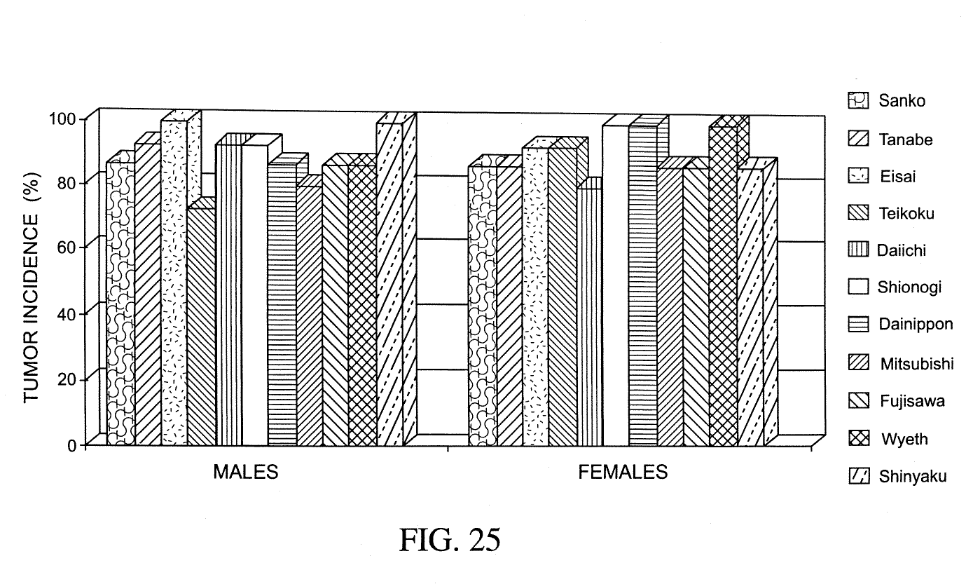

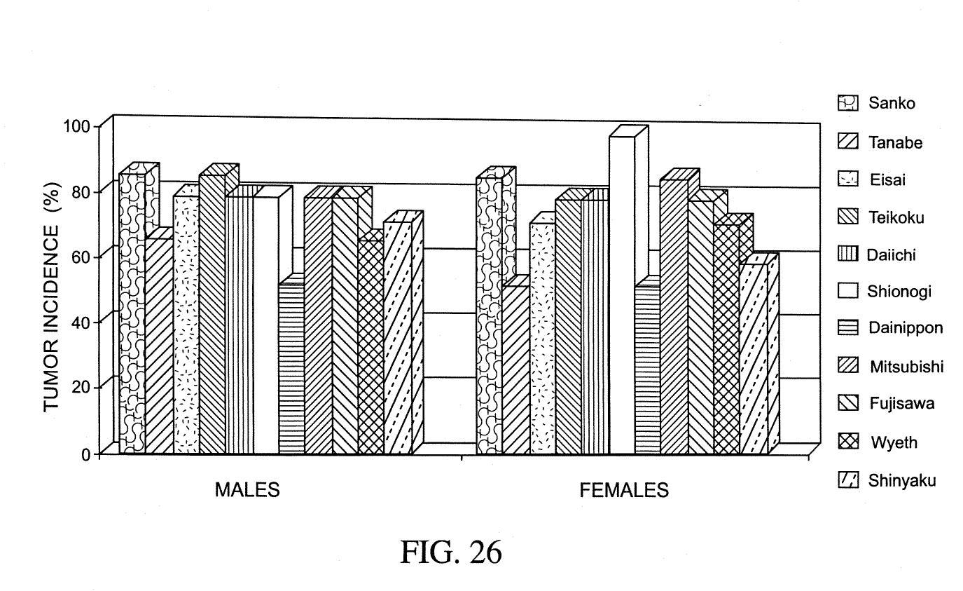

Incidences of tumor, such as squamous cell tumor, in the forestomach, skin, and vagina, carcinoma in the Hardrian gland, adenomas in the lungs, and malignant lymphoma were increased in MNU-treated rasH2 mice. High and consistent incidences of forestomach tumor (FIG. 25) and malignant lymphoma (FIG. 26) were observed among institutions. The overall performance of carcinogenic response of rasH2 mice to MNU as a positive control was judged to be adequate based on qualitatively and quantitatively consistent and robust positive responses for the characteristic spectrum of tumors across multiple institutions (Usui, T., et al., Toxicologic Pathology 29 (Suppl.): 90-108, 2001).

All references cited throughout the specification and the references cited therein are hereby expressly incorporated by reference. While the present invention has been described with reference to the specific embodiments thereof, it should be understood by those skilled in the art that various changes maybe made and equivalents may be substituted without departing from the true spirit and scope of the invention. In addition, many modifications may be made to adapt a particular situation, material, composition of matter, process, and the like. All such modifications are within the scope of the claims appended hereto.

TABLE A

| | | GENER- | | | |
|---|---|---|---|---|---|
| METHODS | GENOTYPE | ATIONS | SEX | AGE | SAMPLES |
| FISH | Tg/+ | N3 | male | 8 w | spleno- |
| | Tg/+ | N20 | male | 6 w | cytes |
| | Tg/Tg | N15 | male | 16 w | |
| SOUTHERN | +/+ | — | male | 8 w | tail DNA |
| BLOT | Tg/+ | N3 | male | 8 w | |
| | Tg/+ | N20 | male | 12 w | |
| | Tg/Tg | N15 | male | 7 w | |
| NORTHERN | +/+ | — | male | 8 w | brain |
| BLOT | Tg/+ | N3 | male | 8 w | total RNA |
| | Tg/+ | N20 | male | 6 w | |
| | Tg/Tg | N15 | male | 16 w | |
| RT-PCR | +/+ | — | male | 8 w | brain, |
| | Tg/+ | N3 | male | 8 w | kidney, |
| | Tg/+ | N20 | male | 6 w | intestine |
| | Tg/Tg | N15 | male | 16 w | total RNA |

What is claimed:

1. A method for planned mass production of transgenic mice or for use as a validated in vivo experimentation system, comprising the steps of:
(a) inducing superovulation in a sexually immature female mutant founder mouse or rat (G0);
(b) fertilizing the superovulating sexually immature female mutant founder mouse or rat;
(c) delivering a first generation mutant mouse or rat (F1) upon completion of the gestation period;
(d) confirming stability of the mutation, genotype, phenotype, and identity of genetic background in the first generation mutant mouse or rat (F1); and
(e) repeating steps (a)-(c) with all further generations of mutant mice or rats for at least twenty generations,
wherein in each step the genetic, microbiological and environmental factors are standardized and kept strictly identical for all mice or rats,
wherein the mutants in each generation are fertilized only if the scheduled genetic monitoring and spot check confirmed that the mutation is stable, and the genotype, phenotype, and genetic background are identical with the genotype, phenotype, and genetic background, respectively of the mutant founder mouse or rat;
(f) determining and standardizing the experimental conditions for the intended use of said transgenic mice or rats; and
(g) validating the transgenic mice or rats as an in vivo experimentation system by periodic monitoring according to a predetermined schedule to verify that their pattern of performance is consistent and uniform in a physiological response relevant to the intended use under the experimental conditions.

2. The method of claim 1 wherein fertilization is performed by natural mating of said female founder mouse or rat with a male mouse or rat, respectively.

3. The method of claim 1 wherein fertilization is performed by
(b.1) subjecting an oocyte obtained from the superovulating premature mutant founder mouse or rat to in vitro fertilization;
(b.2) culturing the fertilized oocyte in vitro to an early embryonic stage; and
(b.3) introducing the embryo into a recipient mouse or rat.

4. The method of claim 3 wherein in step (b.2) said fertilized oocyte is cultured to a two-cell embryonic stage.

5. The method of claim 3 wherein said early embryo is stored in an embryo bank prior to introduction into a recipient mouse or rat.

6. The method of claim 5 wherein said early embryo is stored at liquid nitrogen temperature.

7. The method of claim 1 wherein transgenic mice are produced.

8. (The method of claim 8 wherein the transgenic founder mouse is three to four weeks old at the time of achieving superovulation.

9. The method of claim 8 wherein said transgenic founder mouse is four weeks old at the time of achieving superovulation.

10. The method of claim 8 wherein superovulation is induced by pregnant mare serum gonadotropin (PMSG) and human chorionic gonadotropin (hCG).

11. The method of claim 1 wherein said scheduled genetic monitoring includes monitoring of one or more genes in the genetic background.

12. The method of claim 1 wherein said environmental factors include factors of the developmental and proximate environment.

13. The method of claim 36 wherein the strain mated with the founder mouse or rat to produce the F1 mutant is selected based upon sensitivity to said human disease and the reproductive index of said strain.

14. The method of claim 22 wherein the genetic background is widened in order to achieve widened genetic diversity.

15. The method of claim 22 wherein the usefulness of the selected background strain in modeling a target disease is validated before final selection.

16. The method of claim 8 wherein said transgenic mouse is a Tg-rasH2 mouse, carrying the human c-Ha-ras transgene.

17. The method of claim 29 wherein in step (g) said Tg-rasH2 mouse is validated as an in vivo experimentation system for toxicology and carcinogenicity testing.

18. The method of claim 30 wherein said Tg-rasH2 mouse is validated as an in vivo experimentation system for carcinogenicity testing by administering to said mouse a candidate carcinogenic compound, and determining the carcinogenicity of said compound.

19. The method of claim 1 wherein said use is a model of a human disease
